(19) United States
(12) Patent Application Publication
(10) Pub. No.: US 2003/0037354 A1
(43) Pub. Date: Feb. 20, 2003

(54) Animal model with disrupted Fgf14 gene

(76) Inventors: David M. Ornitz, St. Louis, MO (US); Qing Wang, St. Louis, MO (US); Mark E. Bardgett, Fort Thomas, KY (US); Michael Wong, St. Louis, MO (US); David F. Wozniak, St. Louis, MO (US); Junyang Lou, St. Louis, MO (US); Kelvin Yamada, St. Louis, MO (US)

Correspondence Address: Scott J. Meyer, Pharmacia Corp., 04B, 800 N. Lindbergh Blvd., St. Louis, MO, 63167, US

(21) Appl. No.: 10/206,452

(22) Filed: Jul. 26, 2002

Related U.S. Application Data
(60) Provisional application No. 60/309,687, filed on Aug. 2, 2001.

(51) Int. Cl. A01K 67/027

(52) U.S. Cl. 800/18; 800/21

(57) ABSTRACT

The disclosure describes a unique animal model which is useful for studying the role of FGF14 in the central nervous system (CNS) and testing of potential drugs for treatment of CNS diseases. To provide this animal model, the Fgf14 gene is disrupted in mice by replacing the second and third exons with β-galactosidase. Neuropharmacological studies are disclosed which show that the Fgf14 deficient mice have disrupted striatal-nigra and striatal-pallidal pathways resulting in increased excitatory input to the cortex. The paroxysmal hyperkinetic disorder in Fgf14 deficient mice phenocopies a form of dystonia, a disease often associated with dysfunction of the putamen.

This application claims the benefit of U.S. patent application Ser. No. 60/307,687, filed Aug. 2, 2001.

### FIELD OF THE INVENTION

The present invention relates to the field of fibroblast growth factor related molecules and, more particularly, to fibroblast growth factor 14 (FGF14) and an animal model for studying the role of FGF14 in the central nervous system (CNS) and for testing of potential drugs for treatment of CNS diseases.

### BACKGROUND OF THE INVENTION

Fibroblast growth factors (FGFs) are essential molecules for mammalian development, wound healing and, when inappropriately expressed, cancer. Engineered mutations in the genes encoding several FGFs and FGF receptors (FGFRs) result in developmental defects and/or embryonic lethality. Additionally, point mutations in the genes encoding FGFRs 1, 2 and 3 result in human hereditary craniofacial and skeletal dysplasias.

The first known FGF discovered in the 1970s, now known as FGF2, had an activity that stimulated the proliferation of 3T3 fibroblasts. The FGF family has subsequently grown to include some 22 members of structurally related proteins.

The generation of gene knockouts (e.g., in mice) provides a means for generating defects and then observing their effects on phenotype. For example, as described in U.S. Pat. No. 6,136,040, a transgenic knockout mouse whose genome comprises an introduced null mutation in the Fgf9 gene exhibited under-developed lungs and dilated atrium in 14.5 day old embryos. FGF14 belongs to a distinct branch of the FGF family with heretofore unknown function. FGF14 is widely expressed in the developing central nervous system and in the adult brain. Two isoforms of FGF14, namely FGF14-1a and FGF14-1b, have been cloned which differ only in the first ˜70 N-terminal amino acids, as described by Wang, McEwen and Ornitz, Mech. Develop., Vol. 90, pp. 283-287, (2000). The full 252 amino acid sequence of FGF14-1b is described in U.S. Pat. Nos. 5,773,252 and 6,013,477, although it is erroneously designated FGF15 in these patents. The full 247 amino acid sequence of FGF14-1a is described by Smallwood, et al., Proc. Natl. Acad. Sci. USA, Vol. 93, pp. 9850-9857, (1996).

In particular, FGF14 belongs to a unique subfamily of the known FGFs. This subfamily consists of FGFs 11 to 14, formerly termed fibroblast growth factor homologous factors 1-4 (FHF1-4) (Ref. 47, 53). They share less than 30% amino acid identity with other FGFs, but retain core conserved amino acid residues in exons 2 and 3. They differ from other FGFs in that they apparently do not bind to any of the four known FGF receptors. Heretofore, their biological function remained elusive. FGFs 11-14 share 58-71% amino acid sequence identity and lack a secretory signal peptide (Ref. 53). Multiple splice forms of FGF11-14 have been identified, which only differ in the first exon. The alternative first exons appear to encode dominant protein trafficking signals that are necessary and sufficient to target the protein to different subcellular compartments (Refs. 42, 53, 63, 71). The two forms of FGF14, FGF14-1a and FGF14-1b are localized in the nucleus and cytoplasm, respectively (Ref. 63).

The activity of classical FGFs is regulated in part by cellular localization and export. Many members of the FGF family are efficiently secreted into the extracellular space and function through transmembrane FGF receptors. However, FGFs 1 and 2 lack a consensus signal peptide for secretion but nevertheless can be found in the extracellular matrix of normal cells. It has been shown that FGF1 and FGF2 can also be released when cells are stressed (Ref. 37, 12). In addition, FGF1 and FGF2 can be localized in the nucleus of certain cell types and some studies suggest that nuclear localization of FGF1 is sufficient to trigger DNA synthesis independent of the FGF receptor (Ref. 31, 37). In neurons, exogenous FGF2 can be anterogradely transported along axons in a receptor dependent manner (Ref. 15).

Fgf14 is expressed in the developing central nervous system in mice. At E12.5, Fgf14 is localized in the ventral lining of the third and the fourth ventricles, the supraoptic and septal areas, and spinal cord (Ref. 63). At this stage, Fgf14 is excluded from the neuroepithelium and is localized in the subventricular zone. Fgf14 is localized in the ventral two thirds of the developing spinal cord including the floor plate. Fgf14 is also expressed in several non-neuronal tissues including the thymic primordium and major arteries. In adult mice the cytoplasmic localized form of Fgf14 (Fgf14-1b) is the predominant form expressed in the brain (Ref. 43, 62, 71). The expression pattern of Fgf14 suggests the objective of investigating its role in both neuronal development and adult brain physiology.

Fgfs11-14 have overlapping but non-identical expression patterns, which suggest functional redundancy (Ref. 53). For example, Fgf11, 12, 13 and 14 are all highly expressed in the developing spinal cord at E12.5-E13.5. In contrast to the ventral expression pattern of Fgf14, Fgf12 is expressed throughout the spinal cord with an increasing dorsal to ventral gradient, and Fgf13 is expressed in the ventral-lateral horns of the motor columns and in the dorsal root ganglia (Ref. 25, 63). In adult brains, Fgfs11-14 are widely expressed in the cortex, hippocampus and cerebellum (Ref. 53).

BRIEF DESCRIPTION OF THE INVENTION

In accordance with the present invention a unique non-human vertebrate animal model is provided for studying the role of FGF14 in the central nervous system and for testing of potential drugs for treatment of CNS diseases. To provide a preferred animal model, a null mutation in the mouse Fgf14 gene was engineered. This transgenic knockout mouse was produced by disrupting the Fgf14 gene by replacing the second and third exons with β-galactosidase cDNA.

In a preferred embodiment of the invention, the Fgf14 gene was disrupted by homologous introduction of an Fgf14 N^{N-βGal} allele. This allele abolished FGF14 expression and generated a fusion protein (FGF14N-βgal) between the first exon of FGF14 and β-galactosidase. FGF14N-βgal was localized in the basal ganglia and cerebellum, regions of the brain that regulate motor learning and coordination.

Strikingly, FGF14N-βgal was efficiently trafficked into neuronal processes. Surprisingly and unexpectedly, homozygous FGF14 deficient mice were viable, fertile and anatomically normal, but displayed ataxia and a paroxysmal hyperkinetic dyskinesia beginning at three weeks of age. These motor abnormalities are associated with dysfunction of the basal ganglia system and resemble several human dystonia syndromes. Accordingly, the knockout mouse described herein provides a unique animal model for studying the role of FGF14 activity in human CNS diseases, particularly ataxia and paroxysmal dyskinesis.

Neuropharmacological studies are described herein which demonstrate that the Fgf14 deficient mice have reduced behavioral and transcriptional responses to dopamine agonists. They have disrupted striatal-nigra and striatal-pallidal pathways resulting in increased excitatory input to the cortex. The paroxysmal hyperkinetic movement disorder in Fgf14 deficient mice phenocopies a form of dystonia, a disease often associated with dysfunction of the putamen.

In accordance with these studies, FGF14 is shown to be an important molecule for neuronal signaling. FGF14 is thus believed to be important for: (a) postsynaptic signaling and functions downstream of dopamine receptors in the basal ganglia, and (b) presynaptic signaling and regulating axonal trafficking or neurotransmitter release.

FGF14 binds to a scaffold type molecule called JIP2/IB2 and allows this molecule to bind members of the MAPK cascade (Ref. 51). IB1 and IB2 have been further described as binding to kinesin (Ref. 59). These published findings support a role for FGF14 in the axon transport system or in biochemical activities at the synapse. According to the present invention, an alternate pathway for axon trafficking is provided, whereby the lb amino terminus of FGF14 (FGF14-GFB) alone is sufficient to target the expression of a heterologous protein (β-galactosidase) to axons. That is, when the amino terminus of FGF14-1b is fused to β-galactosidase the FGF-N-β-gal protein is efficiently trafficked down axons in vivo. Thus, the amino terminus of FGF14-1b is itself sufficient for axonal targeting and potentially acts independent of interactions with JIP1/IB2 and kinesin.

In further support of the role for FGF14 in the axon transport system, Neuro2a cells were transfected with FGF14-GFB and have shown that the protein is present in the cytosol and neurite projections and can colocalize with the synaptic vesicle marker, SV2. These results show that FGF14 is efficiently trafficked into axons.

In general, the animal model thus is useful for testing the activity of potential drugs for CNS diseases and disorders such as ataxia, obesity, anxiety/depression (based on localization only), hippocampal hyperexcitability, seizure disorders, bipolar, migraine, cognition (including schizophrenia, ADHD, AD), increased sensitivity to cocaine-induced seizures, increased long term potentiation, substance abuse, movement disorders, Parkinson's disease, Huntington's diseases, dystonia/dyskinesia, ALS (related to cell trafficking) and neuromuscular disorders.

Test compounds can be screened for activity against CNS diseases by conventional in vivo endpoint assays for the diseases by utilizing the FGF14 deficient mouse in the assay, or by treating the Fgf14 deficient mouse directly with the test compound and determining whether there is improvement in CNS function of the mouse, or by developing Fgf14 deficient cell lines from these mice and assaying subcellular localization of FGF14N-βgal in response to test compounds.

Screening of test compounds for activity against CNS diseases and disorders by utilization of animal models and neurocellular components can be carried out in accordance with well known procedures by the person skilled in the art. See, e.g., U.S. Pat. No. 4,707,491 and Klunk, et al., Molecular Pharmacology. Vol. 22, pp. 438-443 (1982), for screening of test compounds in mice, guinea pigs, and hippocampal slices, for example: Testing of behavioral and electrophysiological effects in mice such as drowsiness, hyperexcitability, ataxia and seizures; standard rotorod procedures for neurotoxicity; effects on the EEG of paralyzed-ventilated guinea pigs; effects on seizures induced by pentylenetetrazol (PTZ); and effects on electrical activity of incubated hippocampal slices.

See also, U.S. Pat. No. 5,206,415 and Rodgers-Neame, et al., Molecular Pharmacology, Vol. 42, pp. 952-957 (1992), for screening of test compounds to alter gamma-aminobutyric acid (GABA) responses by examining effects on GABA-noted chloride currents in cultured rat hippocampal neurons. GABA is believed to be the major inhibitory neurotransmitted in the vertebrate CNS.

Strategies for phenotypic analysis in mutant mice for CNS diseases and disorders have been reviewed recently by Lisa M. Gold, Psychopharmacology, Vol. 146, pp. 2-4 (1999). Several conventional methods of screening described by the author which can be used with the Fgf14 deficient mouse of the present invention are, e.g.,

The analysis can include monitoring of simple reflexes (inhibition and emergence) to provide information about the pattern of function of a particular system.

To assess anxiety, basal reactivity to stressors and novelty/exploration can be characterized in the plus-maze, black/white box and light/dark emergence paradigms.

Monitoring of basic physiologic parameters, e.g., food/water intake and body weight gain, are important aspects of screening programs.

Motor activity can be assessed by rotorod balancing and monitoring for cerebellar dysfunction, vestibular problems, general muscle weakness and catalepsy.

Cognition can be examined in behavioral tasks that require integration of motivational, sensory, learning, memory and/or motor processes.

Sensory thresholds, e.g., pain thresholds, can be useful for assessing the sensitivity to normal stimulation and under drug conditions.

Human movement disorders such as Parkinson's disease, Huntington's diseases and dystonia are caused by brain lesions that effectively disrupt the balance between two pathways. That is, in current models of basal ganglia thalamocortical circuitry, the caudate-putamen (CPu), receives input from the cortex and projects through two pathways to the substantia nigra reticulata (SNr) and the internal segment of the globus pallidus (Gpi) (Ref. 57) (FIG. 10A, herein). The direct pathway consists of striatal neurons projecting directly to the Gpi/SNr. These neurons express D1 class dopamine receptors (D1R) and provide inhibitory input to the Gpi/SNr. The striatal neurons that primarily express D2 class dopamine receptors (D2R) provide net excitatory input to the Gpi/SNr via inhibitory projections to the external segment of the globus pallidus (Gpe) and subthalamic nucleus (STN). This pathway is thus referred to as the indirect pathway. The Gpi/SNr integrates input from the direct and indirect pathways and provides an inhibitory signal to the thalamus. The balance of the net input from the direct and indirect pathways are critical for the planning, initiation and execution of movements (Ref. 57, 61, 46, 65).

Neurodegenerative disorders such as Huntington's diseases can also be characterized by symptoms such as neuronal protein aggregates as described by Yamamoto et al., Cell, Vol. 101, pp. 57-66 (2000), or motor deficits observed in a variety of fore- and hind-limb coordination, balance and sensorimotor gating as described by Carter et al., J. Neuroscience, Vol. 19, pp. 3248-3257 (1999). Parkinson's disease can also be characterized by comparative gait analysis as described by Stolze et al., J. Neurol. Neurosurg. Psychiatry, Vol. 70, pp. 289-297 (2001), or by motor initiation and execution as described by Montgomery et al., Movement Disorders, Vol. 15, pp. 511-515 (2000).

Latency to fore- and hindlimb clonus, clonic running seizure and jumping bouncing seizure also are useful end points for testing susceptibility to behavioral seizures induced by cocaine as described by Golden et al., Neuropsychopharmacology, Vol. 24, pp. 291-299 (2001). Effect upon severity of PTZ (pentylenetetrazol)-induced convulsions is a common end point for seizure analysis as described by Mazarati et al., J. Neuroscience, Vol. 20, pp. 6276-6281 (2000).

The utility of the disclosed animal model for studying the role of FGF14 in the foregoing CNS diseases and disorders and for testing potential drugs for treatment thereof is evident from the neuropharmacological studies described herein.

In the illustrative specific working EXAMPLES hereinbelow, the inventors describe with respect to the FGF14 deficient mouse detailed methods and results for the following tests:

Sensorimotor tests;

Dopamine and metabolite measurement;

Cocaine-induced seizure activity;

Pentylenetetrazol (PTZ)-induced seizure activity;

Basal ganglia thalamocortical circuitry;

Learning/memory tests;

Synaptic localization tests; and

Electroencephalography.

The results of the sensorimotor tests were indicative of ataxia and paroxysmal dyskinesia.

Measurement of dopamine and metabolites demonstrated comparable levels with control mice, but the Fgf14 deficient mice showed abnormal responses to dopamine agonists and had a reduced threshold to seizure induced by cocaine.

The basal ganglia thalamocortical circuitry in the Fgf14 deficient mice showed an imbalance between direct (D1R) and indirect (D2R) signaling pathways. As stated above, human movement disorders such as Parkinson's disease, Huntington's diseases and dystonia are caused by brain lesions that effectively disrupt the balance between these pathways.

In the learning/memory tests the Fgf14 deficient mice exhibited significantly more freezing behavior than the control mice.

In the synaptic localization tests the Fgf14 deficient mice were shown to cause elevated excitability in the hippocampal dentate gyrus.

Although specific methods of screening and endpoint assay are described herein, it will be appreciated that the invention is not limited to these specific methods. Reference can be had by the person skilled in the art to well-known texts, treatises and scientific publications on methods for endpoint assays for CNS diseases. In particular, Crawley and Paylor, Hormones and Behavior, Vol. 31, pp. 197-211 (1997), provide an excellent description of test battery and constellations of specific paradigms to investigate the behavioral phenotypes of transgenic and knockout mice. The authors describe a series of conventional neurological and neuropsychological tests which are effectively used as a first screen for behavioral abnormalities in mutant mice, and a series of specific paradigms, including learning and memory, feeding, pain/analgesia, aggression, reproductive, anxiety, depression, schizophrenia, and drug abuse models. Lists of well-known, published references with extensive descriptions of the methods for each of the behavioral paradigms are provided by said authors. Any of these conventional endpoint assays can be used with the Fgf14 deficient animal model as described herein.

The Fgf14 deficient animal or cell line of this invention can be made by disrupting the Fgf14 gene by homologous recombination. The Fgf14 gene is preferably disrupted by homologous introduction of an Fgf14N-βGal allele in said gene. This can be done by insertion of β-galactosidase CDNA in frame into exon 2.

A preferred method of making the homozygous transgenic knockout mouse of this invention comprises the steps of:

(A) deleting the second and third exons of the Fgf14 gene and replacing with β-galactosidase by homologous recombination in mouse embryonic stem cells,

(B) introducing said embryonic stem cells into a mouse blastocyst and transplanting said blastocyst into a pseudopregnant mouse,

(C) allowing said blastocyst to develop into a chimeric mouse,

(D) breeding said chimeric mouse to produce offspring, and

(E) screening said offspring to identify a homozygous transgenic knockout mouse whose genome comprises deletion of the second and third exons of the Fgf14 gene, and wherein said mouse exhibits ataxia and paroxysmal hyperkinetic dyskinesia.

### DETAILED DESCRIPTION OF THE INVENTION

While the specification concludes with claims particularly pointing out and distinctly claiming the subject matter regarded as forming the invention, it is believed that the invention will be better understood from the following preferred embodiments of the invention taken in conjunction with the accompanying drawings.

### BRIEF DESCRIPTION OF THE DRAWINGS

The file of this patent contains at least one drawing executed in color. Copies of this patent with color drawing(s) will be provided by the Patent and Trademark Office upon request and payment of the necessary fee.

FIG. 1 shows: Targeted disruption of Fgf14 and growth characteristics of Fgf14^{N-βGal/N -βGal} mice.

(A) Structure of the Fgf14 genomic locus (top), targeting vector (middle), and targeted allele (bottom). The coding exons are represented by black boxes. The location of PolII-Neo-loxP gene (solid bar) and β-galactosidase (β-gal) gene (open bar) are indicated in the targeting vector and mutant allele. The location of Southern blot probes are shown. E, EcoRI; X, XbaI; B, BamHI; Rv, EcoRV; S, SalI.

(B) Southern blot of genomic DNA from targeted ES clones (lane 1 and 2) and a wildtype ES clone (lane 3), hybridized with 5′ probe and 3′ genomic probes. To detect 5′ homologous recombination genomic DNA was digested with EcoRI. The wildtype allele yields a 9 kb band and the targeted allele yields a 7.5 kb band. To detect 3′ homologous recombination genomic DNA was digested with EcoRV. The wildtype allele yields a 9 kb band and the targeted allele yields a 4.5 kb band.

(C) PCR genotyping of wildtype (+/+), heterozygous (+/−) and homozygous (−/−) Fgf14^{N-βgal} mice. The 255 bp (primers QW127 and QW128) and 350 bp (primers QW127 and QW129) PCR products represent the wildtype and the targeted alleles, respectively.

(D) Northern blot analysis of RNA from the cerebellum of the wildtype (+/+), heterozygous (−/−) and homozygous (+/+) mice. The left panel shows the ethidium bromide stained gel; the positions of the 18s and 28s rRNA are indicated by arrows. The right panel shows the autoradiography following hybridization with an ³²P-αδCTP labeled Fgf14 cDNA probe.

(E) Representative growth curves of Fgf14^{N-βGal/N-βGal} mice and littermates in a pure 129SV/J genetic background. The insert shows the body weight comparison at age 4-5 months.

(F) Representative growth curve of the Fgf14^{N-βGal/N-βGal} mice and littermates after crossing five generations onto a C57B6/J genetic background. The insert shows the body weight comparison at age 4-5 months. *p<0.05, **p<1.01

FIG. 2 shows: Expression of Fgf14 and Fgf14^{N-βgal} in the adult mousebrain and FGF14 in human brain.

(A-L) Dark field images of in situ hybridization show the expression of Fgf14 (A-F) and Fgf14^{N-βgal} (G-L) in a series of coronal sections of wildtype (A-C and G-I) and Fgf14^{N-βGal/N-βGal} (D-F and J-L) mice.

Representative sections are shown through the forebrain (A,D,G,J), midbrain (B,E,H,K) and hindbrain (C,F,I,L). CPu, caudate putamen; CT, cerebral cortex; DG, dentate gyrus; CB, cerebellum.

(M) Northern blot analysis showing expression of FGF14 in different regions of the human brain. Band intensities were quantified by PhosphorImager analysis and the data was normalized to the expression level in whole brain.

FIG. 3 shows: FGF14N-βgal localization in the adult Fgf14^{N-βGal/N-βGal} brain tissue.

(A) Sagittal whole mount X-gal staining of Fgf14^{N-βGal/N-βGal} brain from a 3 month old mouse. FGF14N-βgal activity is seen in the basal forebrain and cerebellum. Two yellow arrows indicate the relative positions of the two coronal sections shown in panels (B) and (C).

(B, C) X-gal staining of frozen coronal sections show FGF14N-βgal activity in the corpus collosum (CC), caudate-putamen (CPu), globus pallidus (GP), dentate gyrus (DG) and substantia nigra pars reticulata (SNr).

(D-F) Axonal trafficking of FGF14N-βgal in the cerebellum. (D) In situ hybridization showing Fgf14 (or Fgf14^{N-βGal/N-βGal}) mRNA expression in the internal granule cell layer (IGL) of the cerebellum.

(E) Localization of FGF14N-βgal activity in the molecular layer (ML).

(F) Localization of βgal activity in the cerebellum of a control mouse in which the cytoplasmic β-galactosidase cDNA was inserted into the GABA_{A} receptor α6 gene (Ref. 27). Note that βgal activity is localized in both the IGL and ML and is excluded from the Purkinje cell (PC) layer.

FIG. 4 shows: Normal brain cytoarchitecture in Fgf14^{N-βGal/N-βGal} mice. Coronal sections through striatum of the wildtype (A and E) and Fgf14^{N-βGal/N-βGal} (B and F) mice, and through the cerebellum of the wildtype (C and G) and Fgf14^{N-βGal/N-βGal} (D and H) mice at 3 month of age.

Sections (A-D) are stained by Luxol Fast blue to show axon myelination.

Sections (E-H) are stained with silver to show the axon tracts.

(I, J) Birth-dating of wildtype (I) and Fgf14^{N-βGal/N-βGal} (J) mice that were labeled in utero with BrdU at embryonic day 13.5 and examined at 1 month of age for cells containing BrdU.

FIG. 5. Sensorimotor evaluation of Fgf14^{N-βGal/N-βGal} mice.

(A) Gait analysis showing forefoot-hindfoot correspondence in a wildtype (+/+) mouse and lack of forefoot-hindfoot correspondence in an Fgf14^{N-βGal/N-βGal} mouse.

(B) Rotorod evaluation of wildtype (n=6) and Fgf14^{N-βGal/N-βGal} mice (n=10) (mixed 129SV/J/C57B6/J genetic background). All mice were pre-trained to remain on a constant velocity rotorod (5 rpm) for 60 seconds. Mice were then tested for their ability to remain on an accelerating rotorod over a 5 day period (3 trials per day). Fgf14^{N-βGal/N-βGal} mice were unable to improve performance over the trial period (p<0.001).

(C) Screen grip test. Mice were placed on a 60° inclined screen. Solid bars represent time on the screen (60 sec maximum). Open bars represent the elapsed time for mice to climb to the top of the screen. Grey bars represent the elapsed time that mice were able to remain on an inverted screen (60 sec maximum). Wildtype mice (n=9), Fgf14^{N-βGal/N-βGal} mice (n=8).

(D) Platform test and

(E) ledge test measures the elapsed time that mice maintain balance on a small platform or ledge.

(F) Walk initiation test. This test measures the amount of time required for mice to move outside of a 21 cm square area in the center of a large smooth surface.

(D-F) Open bar represents wildtype mice (n=9), solid bar represents Fgf14^{N-βGal/N-βGal} mice (n=8). All data expressed as the mean±SEM. Asterisks indicate significant difference between +/+ and −/− groups (student t test). *p<0.05, **p<0.01, ***p<0.001.

FIG. 6. Integrity of dopaminergic neurons in the substantia nigra and striatum and dopamine/dopamine metabolite levels in the ventral striatum.

(A, B) Tyrosine hydroxylase (TH) staining in the substantia nigra pars compacta (SNc) and ventral tegmental area showing similar staining patterns in wildtype (+/+) and Fgf14^{N-βGal/N-βGal} (−/−) mice.

(C) X-gal staining of a Fgf14^{N-βGal/N-βGal} brain section showing FGF14N-βgal localization in the substantia nigra pars reticulata (SNr).

(D, E) TH staining in the caudate putamen (CPu) showing similar staining patterns in wildtype (D) and Fgf14^{N-βGal/N-βGal} (E) mice.

(F) Levels of dopamine (DA) and dopamine metabolites, 3,4-Dihydroxyphenylacetic acid (DOPAC) and Homovanillic acid (HVA) in the ventral striatum.

FIG. 7. Locomotor activity response to stimulant drugs and dopamine receptor agonists.

(A) Cohorts of wildtype (n=9, open bars) and Fgf14^{N-βGal/N-βGal} mice (n=14, solid bars) were treated with saline, cocaine (10 mg/kg) or amphetamine (2 and 4 mg/kg) and assayed for locomotor activity. Fgf14^{N-βGal/N-βGal} mice showed no locomotor response to 10 mg/kg cocaine (p<0.006 relative to +/+mice) and had a reduced locomotor response to both doses of d-amphetamine (p<0.01relative to +/+mice).

(B) The same cohorts of mice were treated with saline, the D1 receptor agonist SK38393 or the D2 receptor agonist quinpirole. Fgf14^{N-βGal/N-βGal} mice lacked a hypo-locomotor response to the D2 agonist, quinpirole (p<0.0002 relative to +/+mice). There was a slight trend towards a significant difference (p=0.12) between the groups after injection of the D1 agonist, SKF38393. All data is expressed as the mean ±SEM. **p<0.01, ***p<0.001.

FIG. 8. Induction of c-FOS nuclear localization in response to cocaine.

Imnunohistochemical detection of c-FOS in wildtype (A, C) and Fgf14^{N-βGal/N-βGal} (B, D) brain tissue 2 hr following intraperitoneal injection of either saline (A, B) or cocaine (40 mg/kg) (C, D).

Following cocaine injection Fgf14^{N-βGal/N-βGal} mice shows diminished levels of c-FOS expression compared to control mice.

(E) Quantification of c-FOS labeled nuclei in the dorsal striatum. After cocaine induction the number of c-FOS positive nuclei in Fgf14^{N-βGal/N-βGal} striatum is significantly lower than that of wildtype controls, *p=0.02 (n=3 for wildtype treated with PBS or cocaine and for Fgf14^{N-βGal/N-βGal} treated with cocaine, n=2 for Fgf14^{N-βGal/N-βGal} treated with PBS), Data is expressed as the mean±SEM. Open bars are wildtype controls, solid bars are Fgf14^{N-βGal/N-βGal} mice. CC, corpus callosum; Cpu, caudate-putamen; CT, cerebral cortex; S, septum.

FIG. 9. Cocaine and PTZ-induced seizure activity.

(A,B) Baseline EEG recordings for wildtype and Fgf14^{N-βGal/N-βGal} mice.

(C,D) EEG recordings following injection of 40 mg/kg cocaine showing epileptiform activity in Fgf14^{N-βGal/N-βGal} mice.

(E and F) EEG recordings following injection of 80 mg/kg cocaine showing epileptiform activity in both wildtype and Fgf14^{N-βGal/N-βGal} mice.

(G) Latency to onset of tonic-clonic seizure following IP injection of PTZ (100 mg/kg). Data is plotted as the mean±SEM, n=8, ** p=0.003.

(H,I) ERK activation in response to treatment with PTZ. (H) Western blot detection of p44/42 MAPK (ERK) and phospho-ERK in wildtype (+/+) and Fgf14^{N-βGal/N-βGal} (−/−) cerebral hemispheres following intraperitoneal injection of PTZ. (I) Quantification of the ratios of the band intensities of p-ERK and total ERK shown in (H) and in additional data. The ratio in wildtype mice was arbitrarily normalized to 1.

No significant difference in ERK activation was observed in the cerebral hemispheres of wildtype (open bars) and Fgf14^{N-βGal/N-βGal} (solid bars) mice injected with saline (n=4 of each genotype). However, following treatment with PTZ, Fgf14^{N-βGal/N-βGal} mice showed significantly higher level of p-ERK compared to littermate controls. Data is plotted as the mean±SEM, (n=7 of each genotype, *p<0.04).

FIG. 10. is a: Schematic diagram showing basal-ganglia-thalamocortical circuitry under normal conditions and in Fgf14^{N-βGal/N-βGal} mice.

(A) Basal-ganglia-thalamocortical circuitry in wildtype mice.

(B) Basal-ganglia-thalamocortical circuitry in Fgf14^{N-βGal/N-βGal} mice.

Fgf14^{N-βGal/N-βGal} mice appear to have an imbalance between the striataonigral (D1R) and striatopallidal (D2R) signaling pathways resulting in increased thalamocortical output. The green lines represent the excitatory neuronal projections and the red lines represent inhibitory projections. The width of the lines represent relative intensity of signaling. The black arrows, ↑ or ↓, indicate pathways that are either hypoactive or hyperactive in Fgf14^{N-βGal/N-βGal} mice. Abbreviations are: CPu, caudate putamen; Gpi, globus pallidus internal segment; GPe, globus pallidus external segment; PPN, pedunculopontine nucleus; STN, subthalamic nucleus; SNc, substantia nigra pars compacta; SNr, substantia nigra pars reticulata; Thal, thalamus.

FIGS. 11 and 12 show: Transfection of Neuro-2a cells with FGF14-GFP.

FIG. 11 shows synaptic localization of FGF14-1b in neuroblastoma (Neuro-2a) cells.

(A). Myc tagged FGF14-1b was transiently transfected into Neuro-2a cells. Inmmunostaining using an antibody against Myc (FITC) shows the cytoplasmic localization of FGF14-1b in undifferentiated Neuro-2a cell.

(B). Transfected Neuro-2a cells were differentiating after 1-2 day retinoic acid treatment. FGF14-1b protein are localized in extending neurites.

(C). In the fully differentiated Neuro-2a cells, FGF14-1b are concentrated in variscosities along the neurites.

(F). Double immunostaining with antibodies against Myc (FITC) and synaptic vesicle protein I (SV2) (Cy3) shows the co-localization of FGF14-1b (D) and SV2 (E).

FIG. 12, FGF14 Expression in the hippocampus.

FIG. 12 shows that lack of FGF14 causes the elevated excitability in the hippocampal dentate gyrus.

(A). FGF14N-βgal protein is localized in the hilus of the dentate gyrus.

(B). The expression of FGF14 mRNA is localized in the dentate gyrus granule cells by in situ hybridization.

(C). Representative dentate granule cell recording in hippocampal slices from the wildtype mice (top panel) and the FGF14 deficient mice (bottom panel). Upon stimulation, the slices from the FGF14 deficient mice (8 out of 12 slices) show multiple spikes compared to the control slices (2 out of 13 slices), p<0.05 by student t test.

FIG. 13, Shows time to death following PTZ-induced seizure.

The time to death of wild type mice (n=8) and FGF14(−/−) mice (n=8) following IP injection of PTZ was measured in two experiments. In Experiment 1 the average age was 82 days, the sex was 50% female and the PTZ dose was 100 mg/kg. In Experiment 2 the average age was 354 days, the sex was 100% female and the PTZ dose was 75 mg/kg. FIG. 13 shows that the wild type mice died within a matter of a few seconds (barely visible black bars at left for each experiment), whereas the FGF14 (−/−) mice survived for more than an hour (mean duration about 4200 and 5700 seconds, shaded bars at the right for each experiment).

In order to illustrate the invention in greater detail, the following specific laboratory examples were carried out. Although specific examples are thus illustrated herein, it will be appreciated that the invention is not limited to these specific, illustrative examples or the details therein.

### EXAMPLE I

### Preparation and Neuropharmacological Testing of Fgf14 Deficient Mice

In this EXAMPLE I the in vivo activity of FGF14 was disrupted by homologous introduction of an Fgf14^{N-}_{β}^{Gal} allele which abolished FGF14 expression and generated a fusion protein (FGF14N-βgal) between the first exon of FGF14 and β-galactosidase. Fgf₄^{N-}_{β}^{Gal} was localized in the basal ganglion and cerebellum, regions of the brain that regulate motor behavior. Strikingly, FGF14N-βgal chimeric protein was efficiently trafficked into neuronal processes. Fgf14 deficient mice were viable, fertile and anatomically normal, but developed ataxia and a paroxysmal hyperkinetic movement disorder.

Neuropharmacological studies in this Example showed that the Fgf14 deficient mice have disrupted striatal-nigral and striatal-pallidal pathways resulting in increased excitatory input to the cortex. The paroxysmal hyperkinetic movement disorder in Fgf14 deficient mice phenocopies a form of dystonia, a disease often associated with dysfunction of the putamen.

### Methods

Gene Targeting and Generation of FGF14 Deficient Mice

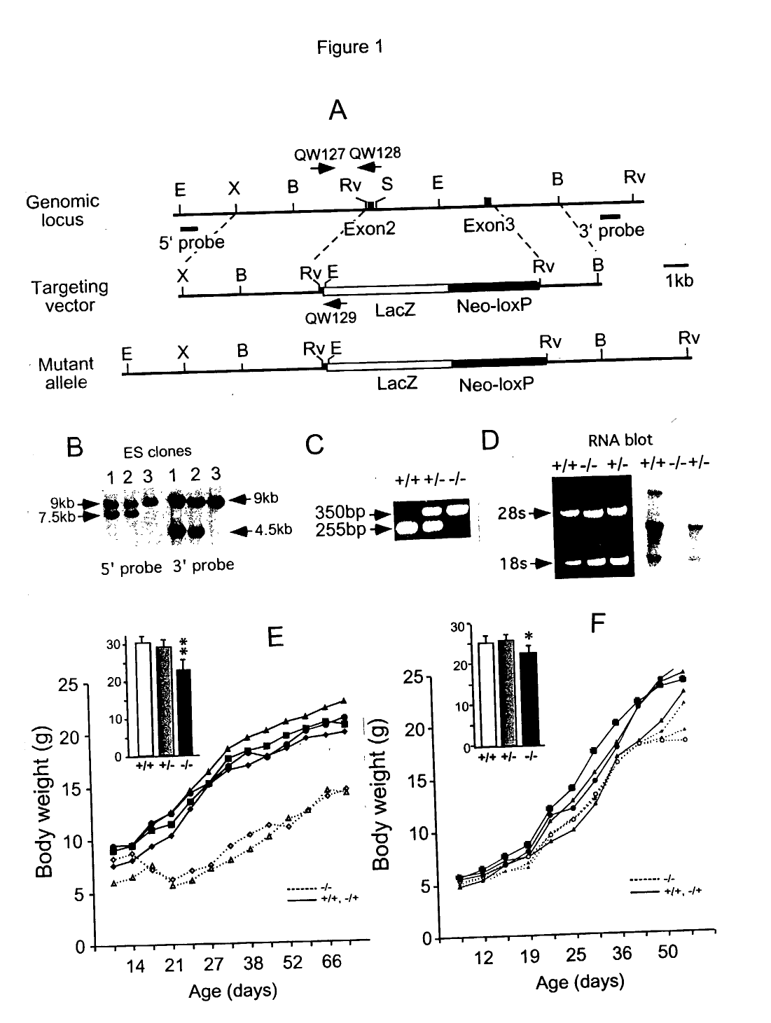

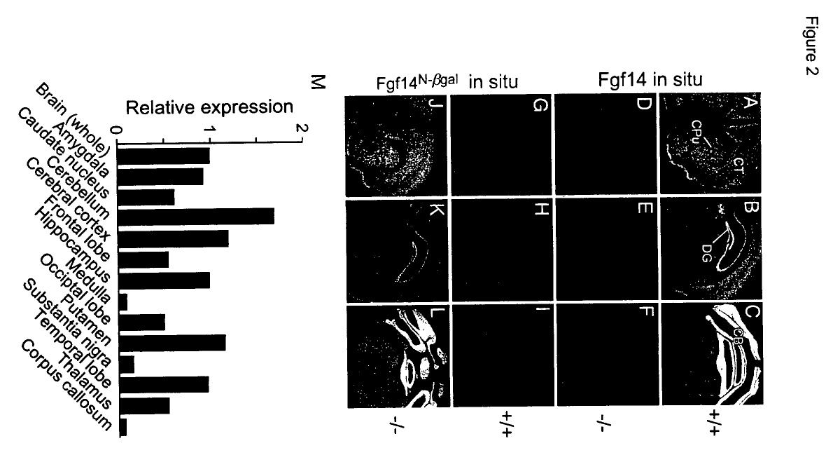

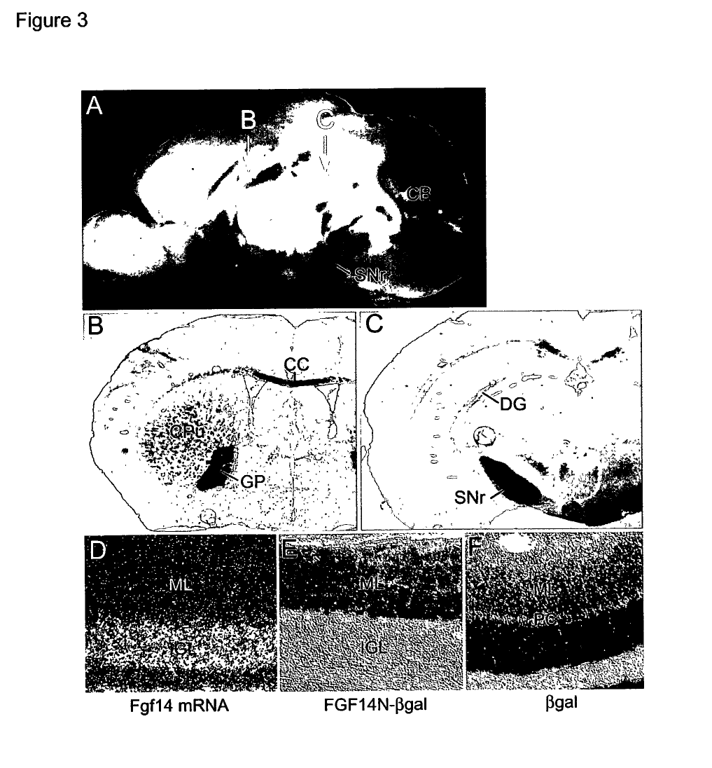

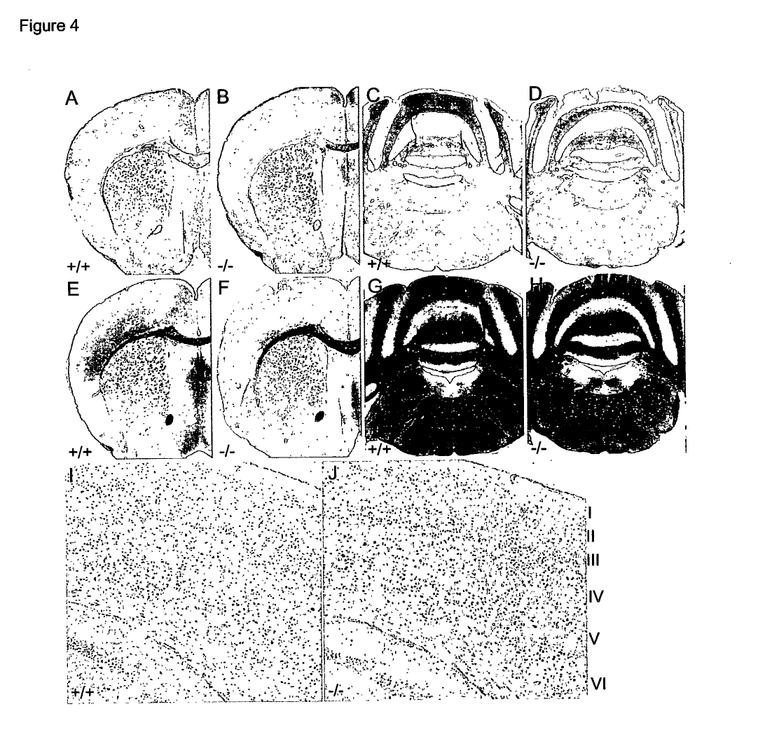

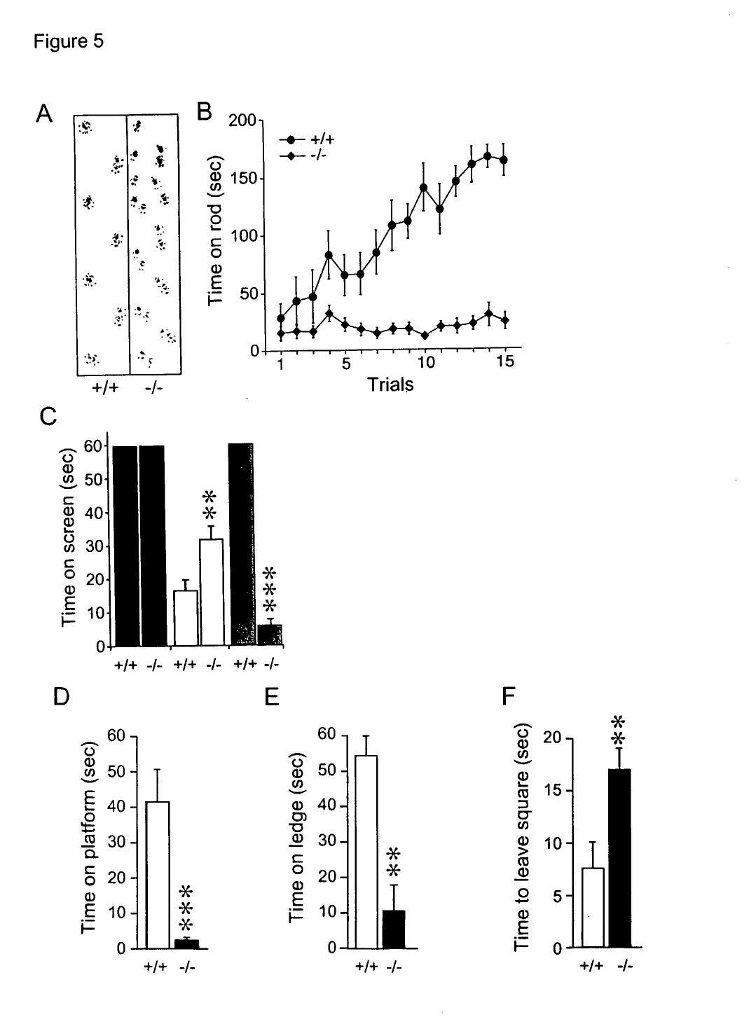

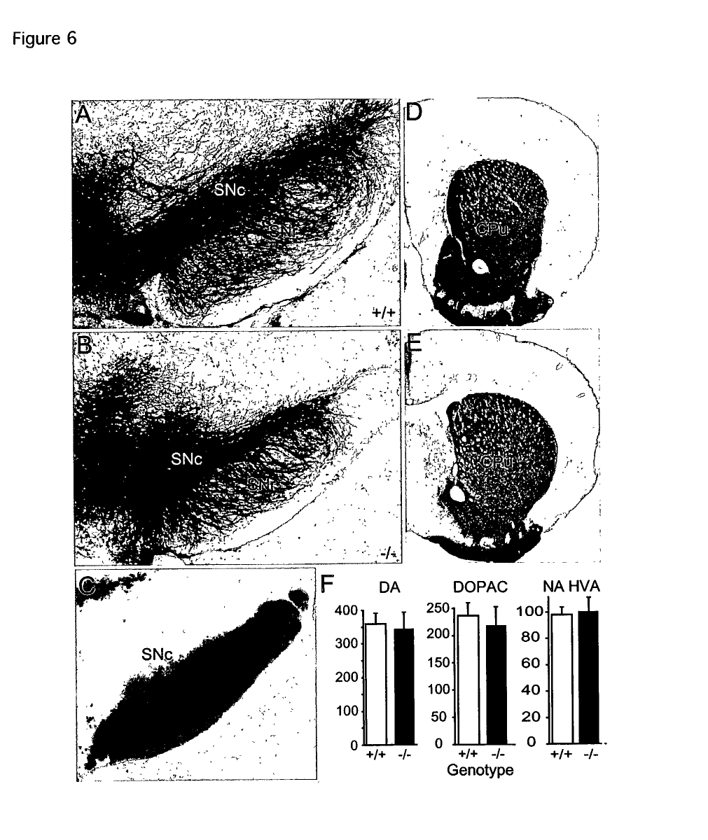

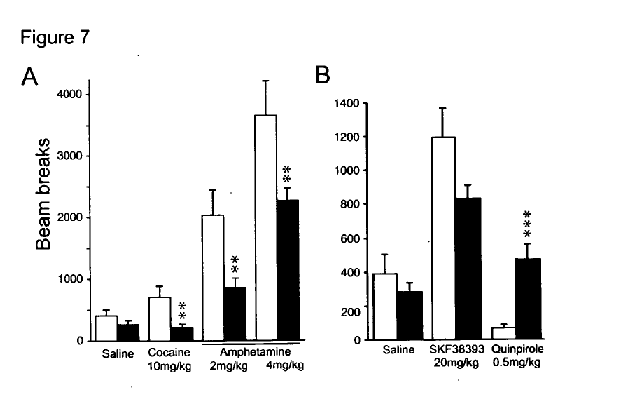

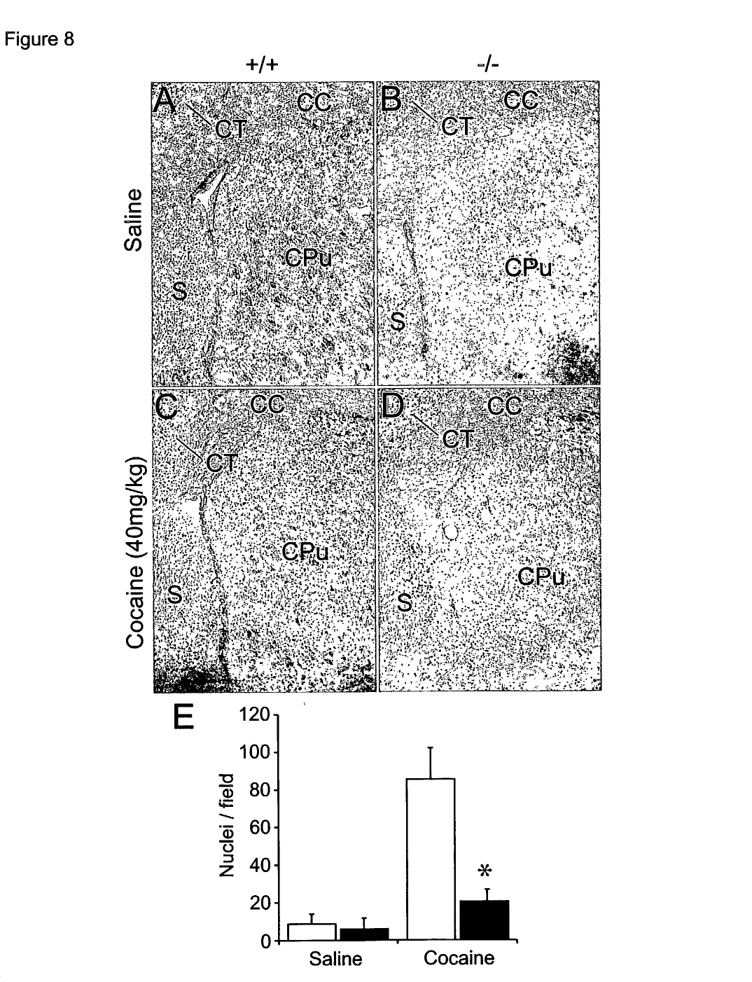

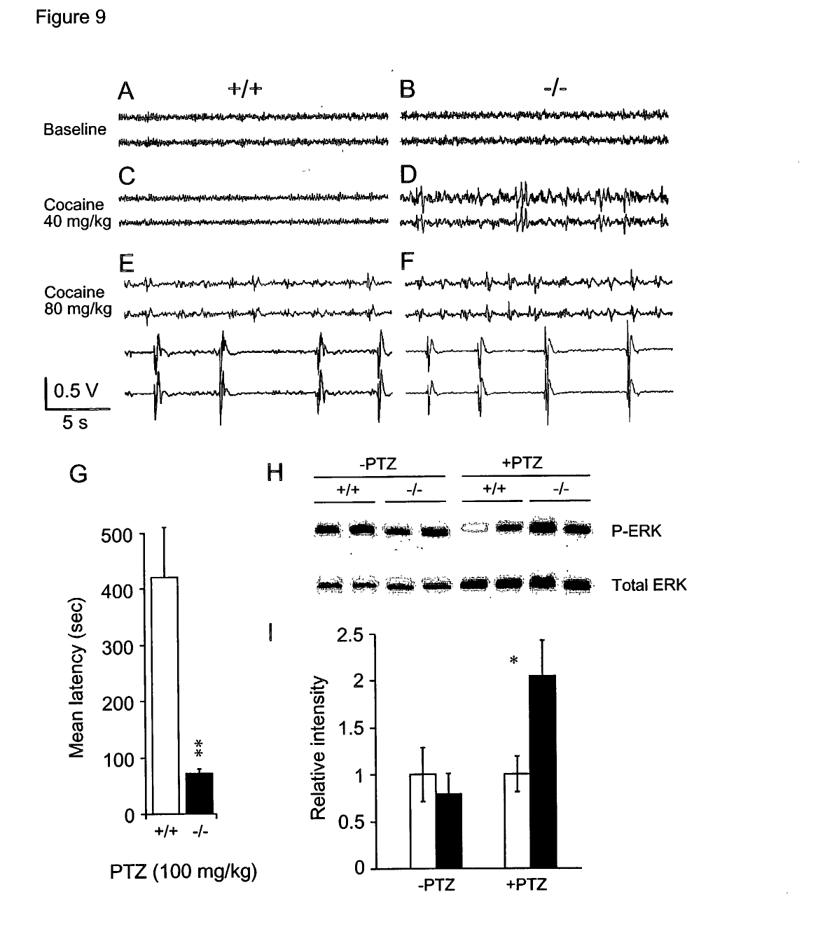

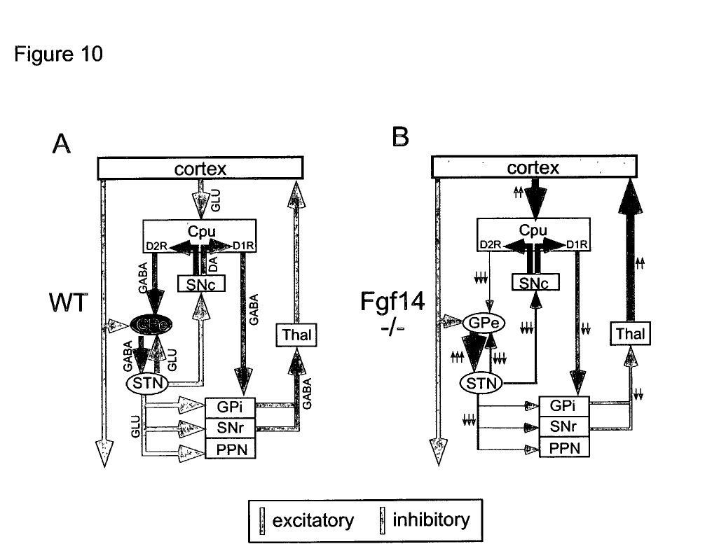

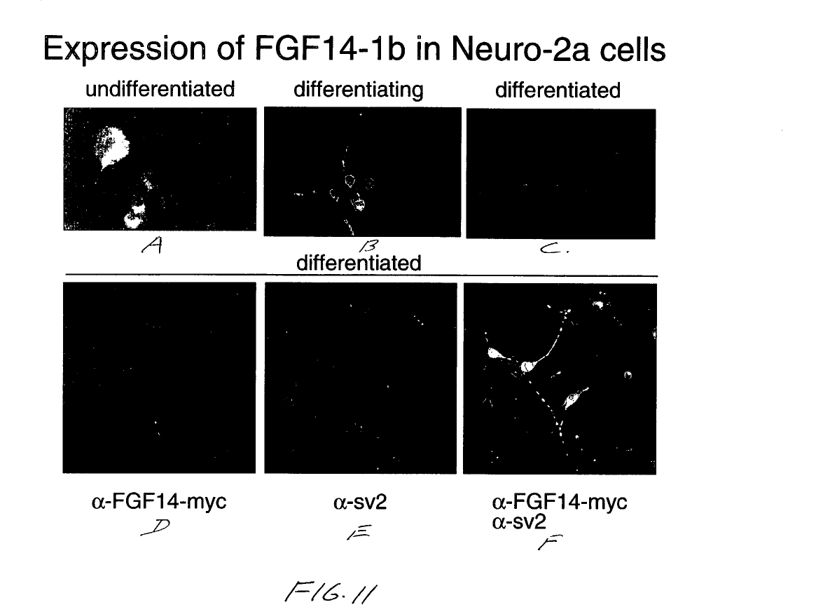

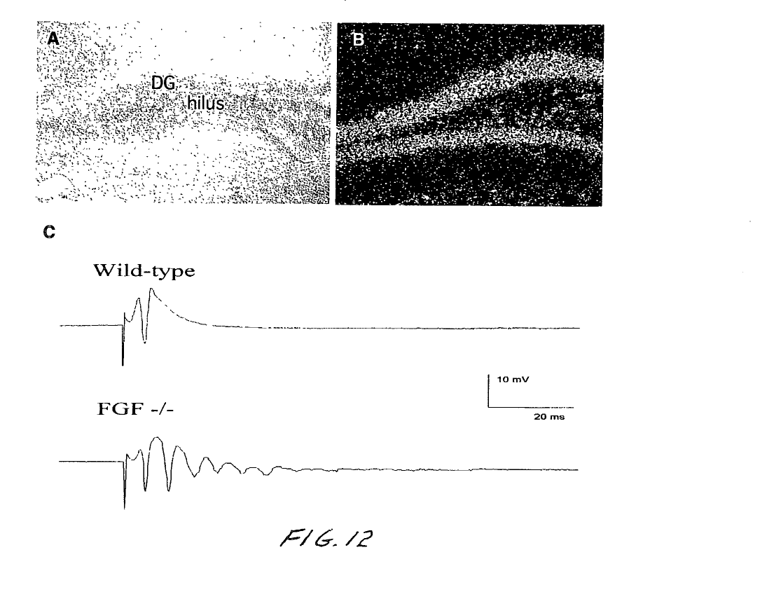

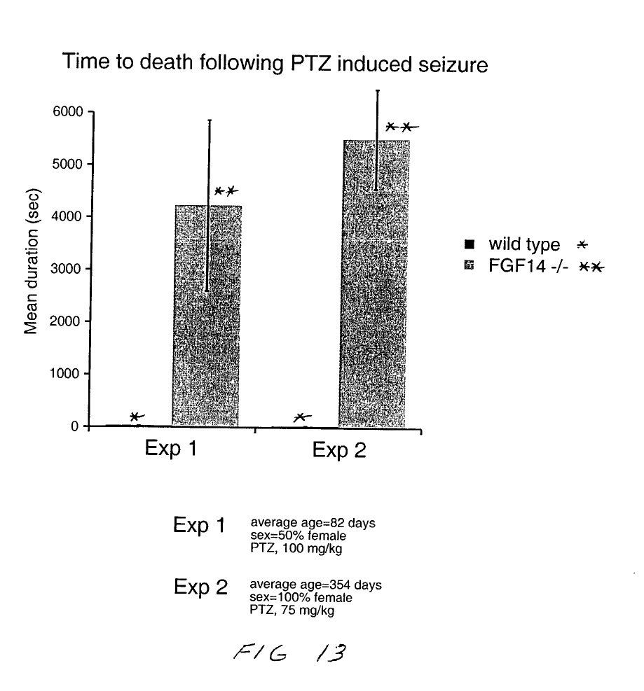

The targeting vector (FIG. 1A) was designed to delete the exon 2 (after the fifth codon), the entire exon 3 and the 3 kb intron between exon 2 and exon 3. A β-galactosidase gene was inserted in frame with the fifth codon of exon 2. The targeting vector contains 4.5 kb of 5′ homologous genomic DNA and 2.7 kb of 3′ homologous genomic DNA flanking the deletion. The Neo selection cassette was cloned downstream of the inserted β-galactosidase gene and is flanked by two loxP sites which can be used for cre-mediated excision. The targeting vector was also flanked by two TK genes. The plasmid was linearized with Sfi1 and electroporated into SM1 embryonic stem (ES) cells and selected with G418 and FIAU. Genomic DNA was isolated from ES cells, digested with EcoRI and hybridized with a 5′ external probe (400 bp PCR fragment) (FIG. 1B) or digested with EcoRV and hybridized to a 3′ external probe (700 bp XbaI fragment). Two correctly targeted clones were identified out of two hundred ES cell colonies surviving selection with G418 and FIAU. These clones were individually injected into blastocysts derived from C57BJ/6 mice. Both clones gave rise to chimeric mice that transmitted the targeted allele into the germline.

Genotyping of progeny was by Southern blot (as described above) or by PCR analysis of tail DNA. The 5′ primer, QW127 (5′-CTAGTTTCATGAAATCCCTATTTC-3′) [SEQ ID NO: 1], hybridizes to the 5′ homologous region. The 3′ primers were QW128 (5′-GCCTTGCCTGCAATATAACCTGGTCAC-3′) [SEQ ID NO: 2] hybridizing to Fgf14 exon 2 for the wildtype allele, and QW129 (5′-CGCTATTACGCCAGCTGGCGAAAG-3′) [SEQ ID NO: 3] which hybridizes to the 5′ region of the β-galactosidase cDNA. PCR conditions were: 30 cycles (95° C. for 30 sec., 55° C. for 1 min., 72° C. for 1 min.), followed 3 min. at 72° C. The PCR assay generated a 255 bp fragment for the wildtype allele, and a 350 bp fragment for the Fgf14^{N-βgal} allele (FIG. 1C).

Northern Blot Hybridization

RNA was isolated for the cerebellum of 21 day old mice using the RNeasy kit (Qiagen). 6.41 μg of total RNA was separated by electrophoresis at (4 V/cm) through a 1% agarose gel containing 0.66 M formaldehyde and transferred to N-Hybond membranes (Amersham) by capillary elution. A 315 bp Fgf14 probe derived from exon2, exon3 and a part of exon4 was labeled with ³²P-αδCTP by the random priming method. Filters were hybridized and washed under high stringency conditions, and exposed to X-ray film. Human brain northern blot was purchased from Clonetech Inc. and hybridized with the 315 bp mouse Fgf14 probe. Mouse and human cDNAs share >95% identity in this region. Band intensity was quantified by scanning densitometry using the NIH image software.

In Situ Hybridization

Brain tissues were harvested from 30 day old mice that were perfused with 4% paraformaldehyde. Frozen sections were acetylated (0.1 triethanolamine, pH 8.0, 0.25% acetic anhydride) for 15 minutes at room temperature, and hybridized at 55° C. overnight (1×10⁶ cpm/ml riboprobe, 50% formamide, 4×SSC, 1× Denhardt's solution, 10% dextran sulfate, 50mM DTT, 500 μg/ml yeast t-RNA and 300 μg/ml denatured hering sperm DNA). Following hybridization, the sections were washed and digested with Rnase A as described previously (Ref. 43). The sections were dehydrated and air dried. Autoradiography was carried out using a 1:1 ratio of NTB emulsion (Kodak) to water. Sections were developed in D-10 developer with Kodak rapid fixer for 5 minutes. The slides were counter stained with Hematoxylin.

Staining for β-galactosidase Activity

FGF14N-βgal and β-galactosidase enzymatic activity was localized in situ by staining with 5-bromo-4-chloro-3-indolyl-β-d-galactoside (X-gal). Three month old mice were perfused with 2% paraformaldehyde. Brains are harvested and embedded in OCT compound and cryostat sectioned at −20° C. Subsequently, sections were stained in 0.1M sodium phosphate at pH=7.0, 2 mM MgC12, 5 mM K₃Fe(CN)₆, 0.01% sodium deoxycholate, 0.02% NP40, 20 mM Tris-HCl and 1 mg/ml of X-gal at 37° C. overnight. Slides were then counter stained with nuclear fast red.

Histology and Immunohistochemistry

Deeply Anesthetized mice were perfused with 4% paraformaldehyde, processed through graded dehydration and embedded in paraffin. Nissl, Luxol Fast Blue, and silver stains were carried out according to (Ref. 52).

For immunohistochemistry, brains were removed from mice perfused with 4% paraformaldehyde and then postfixed for 12 hrs in 4% paraformaldehyde. Tissues were cryoprotected by immersion in 30% sucrose overnight and then were embedded in O.C.T. compound (Tissue-Tek) and placed on dry ice. Frozen sections were cut at 40 μm and stored in PBS/azide solution at 4° C. until used. Sections were incubated in 0.3% H₂O₂ in PBS for 30 min at room temperature. After blocking in 3% normal goat serum in PBS for 30 min, sections were incubated with anti-c-fos antibody (1:30,000, Oncogene Science, Inc.), or anti-tyrosine hydroxylase antibody (1:1,000, Chemicon International, Inc) in blocking solution. Sections were then incubated with the appropriate secondary antibody coupled to horseradish peroxidase. Peroxidase staining was visualized using the Vectastain ABC kit (Vector Laboratories).

Sensorimotor Tests

The sensorimotor battery included the inclined screen, platform, ledge and walking initiation tests performed as described by Schaefer et. al. (Ref. 50).

Inclined and inverted screen test. Each mouse was placed on top of an elevated (47 cm above the floor) wire mesh grid (16 squares per 10 cm) that was inclined to 60°. The wire mesh grid was stretched across a wooden frame that was 15×52 cm. Tape was placed along the perimeter to prevent the mice from climbing around to the back of the apparatus. Each animal was placed in the middle of the screen with its head oriented down and timed for how long it remained on the screen and how long it took to climb to the top of the screen. For the inverted screen test mice were placed as above and then the screen was inverted to 180°. A maximum score of 60 sec was given if an animal did not fall.

Platform test. Each mouse was timed for how long it remained on an elevated (47 cm above the floor) circular platform (1.0 cm thick; 3.0 cm in diameter). A maximum score of 60 sec was assigned if the mouse remained on the platform for the maximum amount of time or if it could climb down on a very thin pole that supported the platform without falling.

Ledge test. Each mouse was timed for how long it could maintain its balance on a 0.75 cm wide Plexiglas ledge without falling (60 sec maximum). A score of 60 sec was also assigned if the mouse traversed the entire length (51 cm) of the Plexiglas ledge and returned to the starting place in <60 sec without falling.

Walking initiation test. Each mouse was placed in the middle of a square outlined by white cloth tape (21×21 cm) on a smooth black surface of a large table top. The time it took each mouse to leave the square (place all four paws outside of the tape) was recorded. The maximum time allowed was 60 sec.

Rotorod test. The rotorod apparatus (Columbus Instrument) was used to measure motor coordination and balance. During the training period, each mouse was placed on a stationary rotorod and then on a rotating rotorod at a constant speed (2.5 rpm). Once each mouse was able to maintain balance for 1 min on three consecutive trials, it proceeded to the next training step. After successful completion of the training, each mouse was tested on the accelerating rotorod (5-20 rpm over 0-180 sec) three trials per day for 5 consecutive days. The mean latency to fall off the rotorod was recorded.

Locomotor activity. For the locomotor activity experiments, all mice were acclimated to the activity cages (28 cm long×17 cm wide×12.5 cm high) for one hour on two consecutive days. For each drug experiment, animals were placed in the activity cages for 30 minutes, before receiving an intraperitoneal injection (cocaine hydrochloride (Sigma) 10 mg/kg, or SKF38393 20 mg/kg (RBI, Natik, Md.)) or a subcutaneous injection (d-amphetamine sulfate (Sigma) 2.0 or 4.0 mg/kg, or quinpirole 2.0 mg/kg (RBI, Natik, Md.)) and were placed back into the activity cages for 55 min. Mice were tested every other day and the order of drug treatment was counterbalanced across days within genotype groups. Locomotor activity was recorded every five minutes by two beams of red light placed at 10 and 19 cm along the length of the activity cage.

Statistical Analyses. The behavioral data were analyzed using analysis of variance (ANOVA) models typically containing one between-subjects variable (Genotype) and one within-subjects variable (e.g., Trials or Test Sessions). Subsequent one-way ANOVAs were used to evaluate main effects. Data from the experiments concerning drug effects on locomotor activity were analyzed by using a two-way analysis of variance (ANOVA) with one between-subjects variable (Drug) being added to the above model. Fisher's protected least squares difference tests were used for these data involving post-hoc comparisons of individual groups. Alpha was set at p<0.05 for all analyses.

Measurement of Dopamine and its Metabolites

Tissue concentrations of dopamine and dopamine metabolites (DOPAC and HVA) were measured by HPLC coupled with electrochemical detection as previously described (Ref. 4). A 2 mm coronal slice of each mouse brain containing the nucleus accumbens and dorsal striatum was isolated with a razor blade. The nucleus accumbens was micro dissected with a 1.6 mm cold stainless steel tube which contained a stylet for punch removal. Punches from the left and right nucleus accumbens were combined in 1.5 ml polypropylene tubes, placed on dry ice, and stored at −80° C. until assayed. Dopmaine, DOPAC and HVA were detected by HPLC using an ESA 5020 guard cell and an ESA 7013 dual electrode analytical cell coupled to ESA Coulochem II detector. All values were derived and analyzed from standard curves for DA, DOPAC, and HVA using a computerized data analysis program (Bechmann System Gold).

Pentylenetetrazol Treatment and ERK Activation Analysis

Pentylenetetrazol (Sigma) was dissolved in saline (10 mg/ml) and injected intraperitoneally at a dose of either 75 or 100 mg/kg. Time to onset of tonic-clonic seizure was recorded. In the first experiment (FIG. 9), the mean age was 82 days and the sex ratio was 50% male/female. In the second experiment, the mean age was 354 days and all animals were female.

For ERK activation, adult (6-23 wk) Fgf14^{N-βGal/N-βGal} and wildtype female mice were injected intraperitoneally with 75 mg/kg PTZ or PBS. On each of three days prior to the day of the experiment, each mouse was twice injected intraperitoneally with PBS to habituate the animals to handling and injections. The animals were sacrificed 90 sec after the experimental injection (or at the onset of status epilepticus). The cerebral hemisphere and cerebellum were removed and homogenized in 500 μl of boiling lysis buffer (1% SDS; 20 μl/ml protease inhibitor cocktail solution (Sigma P8340); and 1 mM Sodium ortho-vanadate). Protein levels in supernatants wee determined by the Bradford method using a protein assay kit (Bio-Rad, Hercules, Calif.). Aliquots of each sample containing 20 μg of protein were electrophoresed on precast 12% Tris-HCI polyacrylamide gels (Bio-Rad). Proteins were visualized with a polyclonal rabbit anti-phospho-p44/42 MAPK (ERK) antibody (1:1000; Cell Signaling, Beverly, Md.) or polyclonal anti-p44/42 ERK (1:1000; Cell Signaling). The images were scanned (AGFA DuoScan), and densitometry data was analyzed using Quantity One Software (Bio-Rad).

Electroencephalography.

Surgical implantation of skull screw electrodes was performed with the mice positioned in a stereotaxic frame under halothane anesthesia. Using sterile technique, the scalp was incised at the midline and the periosteum was scraped free from the skull. Three burr holes were made with a high speed drill for placement of epidural screw electrodes. Two active electrodes were placed over the right and left frontal cortex approximately 1 mm posterior to the bregma. A reference electrode was implanted in the region over the frontal sinus. The electrodes and lead wires were stabilized to the skull with dental cement. The mice were allowed a three-day recovery period before recording. Reference EEG recordings from the right and left frontal regions were performed using a Grass P5 Series AC amplifier with high and low frequency filter settings at 100 and 1 Hz, respectively. The outputs of the amplifiers were digitized and stored using an analog-to-digital converter and Axoscope software.

### Results

Fgf14 Gene Targeting

An Fgf14 gene targeting vector (FIG. 1A) was designed to insert a β-galactosidase (βgal)-Neo cassette into the Fgf14 gene. This design placed the β-galactosidase cDNA in frame with the first 5 codons of exon 2, generating a fusion protein containing the alternatively spliced first exons of FGF14 fused to β-galactosidase (FGF14N-βgal). This resulted in deletion of exons 2 and 3, which encode the core conserved amino acids common to all FGFs (Ref. 63). The targeting construct was electroporated into embryonic stem cells. Screening of 200 clones identified two independent clones in which Fgf14 was disrupted by homologous recombination (FIG. 1B). Both clones were injected into blastocysts to produce chimeric mice, and both were transmitted into the germline (FIG. 1C). The resulting heterozygous mice (Fgf14^{N-βGal/+} or +/−) were indistinguishable from wildtype littermates (wt or +/+). Homozygous mice (Fgf14^{N-βGal/N-βGal} or −/−) were viable and fertile. FGF14 mRNA expression was assessed by Northern blot of total cerebellar RNA using a probe derived from the sequence encoding exons 2 and 3 (FIG. 1D). Fgf4 mRNA was detected in wildtype mice, at reduced levels in heterozygous mice, but was absent in Fgf14^{N-βGal/N-βGal} mice.

During the first two weeks after birth, Fgf14^{N-βGal/N-βGal} pups were indistinguishable from their wildtype littermates. In the third and fourth weeks, the growth of Fgf14^{N-βGal/N-βGal} mice on a pure 129/ SVJ genetic background lagged behind that of wildtype littermates but thereafter maintained a parallel curve. Adult Fgf14^{N-βGal/N-βGal} mice remained ˜30% smaller than littermate controls (n=9 per genotype p<0.01) (FIG. 1E). Fgf14^{N-βGal/+} mice were backcrossed five generations onto the C57B6/J genetic background (G5:C57B6/J). On the G5:C57B6/J genetic background, the size of Fgf14^{N-βGal/N-βGal} mice was similar to that of wildtype littermates until weaning. However, adult Fgf14^{N-βGal/N-βGal} mice failed to attain normal weight and remained 15% smaller than their littermates (n=7 per genotype, p<0.05) (FIG. 1F).

FGF14^{N-βgal} is Expressed in the Cerebellum and Basal Ganglia and is Efficiently Trafficked into Axons

To determine whether Fgf14^{N-βGal} expression recapitulates that of Fgf14, in situ hybridization was carried out using both Fgf14 and β-gal cDNA probes on serial coronal sections from adult wildtype and Fgf14^{N-βGal/N-βGal} brains. This analysis revealed that Fgf14 transcripts were localized in hippocampal and cerebellar granule cells, and were diffusely expressed in the cerebral cortex and striatum (caudate-putamen, CPu) (FIG. 2A-C). Fgf14 transcripts were not detected in Fgf14^{N-βGal/N-βGal} tissue (FIG. 2D-F), consistent with the northern blot data (FIG. 1D). β-galactosidase in situ mRNA expression patterns in Fgf14^{N-βGal/N-βGal} tissue (FIG. 2G-L) were essentially the same as those of Fgf14 in wildtype tissue, suggesting that deletion of the 3 kb intron between exon 2 and exon 3 did not disrupt regulatory elements that control Fgf14 expression. Furthermore, northern blot analysis of FGF14 regional expression in human adult brain (FIG. 2M) showed the highest levels in the cerebellum, and moderate to high levels in the hippocampus, amygdala, cerebral cortex, striatum, and thalamus, consistent with in situ data on adult mouse tissue. The corpus callosum and substantia nigra showed no significant expression. These data demonstrated that the expression pattern of Fgf14 is conserved across species.

To assess the expression pattern of the FGF14N-βgal protein, β-galactosidase enzymatic activity was localized in situ by X-gal staining. FGF14N-βgal was strongly expressed in the cerebellum, and striatum, and moderately expressed in the dorsal cerebral cortex, hippocampus and hypothalamus (FIG. 3A-C). Although globally consistent with the Fgf14 transcript distribution pattern determined by in-situ hybridization and northern blot (FIG. 2), X-gal staining revealed a striking difference. Surprisingly, FGF14N-βgal was efficiently localized in axons, and was barely detectable in neuronal cell bodies. For example, in the cerebellum (FIG. 3D and E), FGF14N-βgal staining is most intense in the molecular layer (ML) where the axons of granule cells extend and fasciculate while Fgf14^{N-βGal} and Fgf14 MRNA are localized in the internal granule cell layer (IGL) which contains the cell bodies of granule cells. In the hippocampus (data not shown), FGF14N-βgal was predominantly localized to the hilus region containing the axons of dentate granule cells whereas Fgf14 mRNA was localized to the granule cell layer. More strikingly, the corpus callosum, a region that only contains a collection of fibers, was intensely stained with X-gal (FIG. 3B, C). In contrast, both in situ hybridization and northern blot showed no expression of Fgf14 mRNA in the corpus callosum. Control transgenic mice which express β-galactosidase in granule cells under the control of GABA receptor α6 subunit regulatory elements (Bahn et al., 1997; Jones et al., 1997) show β-galactosidase activity distributed primarily in the IGL and diffusely in the ML (FIG. 3F). Taken together, these data demonstrate that the first exon of FGF14 is sufficient to direct β-galactosidase into axons, and suggest that FGF14 may itself be trafficked into neuronal processes.

FGF14 is Not Essential for Neuronal Development

Given that Fgf14 is widely expressed in the central nervous system during embryonic development, we hypothesized that loss of FGF14 may result in neuronal malformation. However, Fgf14^{N-βGal/N-βGal} mice are viable and fertile, and have no increased mortality over a 20 month period (Fgf14^{N-βGal/N-Gal}:n=16; wt:n=12). Examination of brain structure using three different histological stains revealed normal organization of cell bodies (Nissl stain, not shown), no evidence of demyelination (luxal fast blue, FIGS. 4A-D) and no axon degeneration (silver stain, FIGS. 4E-H) in Fgf14^{N-βGal/N-βGal} mice at 3-4 month of age. Because Fgf14 is expressed most strongly in the subventricular zone of the developing CNS (Wang et al., 2000a), a region that contains migrating neurons, we examined neuronal migration by BrdU birth-dating (Chae et al., 1997). Pregnant mice were injected with BrdU at day 13 of gestation. Coronal sections, prepared at one month of age from wildtype and Fgf14^{N-βGal/N-βGal} mice, were stained for BrdU to identify the location of post migratory neurons. Sections at the level of the anterior hippocampus identified that most BrdU labeled neurons are localized in cortical layers IV and V in both Fgf14^{N-βGal/N-βGal} mice (FIG. 4I) and in wildtype littermates (FIG. 4J). Thus, neither neuronal migration nor brain histology appeared to be disrupted in mice lacking Fgf4.

Ataxia and Paroxysmal Dyskinesia in Mice Lacking Fgf14

Wildtype mice extend their hindlimbs and digits in response to being suspended by their tail. In contrast, 60% of adult Fgf14^{N-βGal/N-βGal} mice (G5:C57B6/J background, n=10) retracted their hindfeet and clenched their digits when suspended by their tail. The etiology of this behavior has been attributed to both striatal (Yamamoto et al., 2000) and cerebellar origins (Lalonde, 1987), suggesting that Fgf14^{N-βGal/N-βGal} mice may have neurological deficits in these regions. Furthermore, Fgf14^{N-βGal/N-βGal} mice had an ataxic gait with shuffling footprint patterns, a widened stance and lack of forefoot-hindfoot correspondence (FIG. 5A). An accelerating rotorod was used to assess the ability of Fgf14^{N-βGal/N-βGal} mice to coordinate movement (FIG. 5B). Note the general superior performance of control mice relative to the Fgf14^{N-βGal/N-βGal} mice. Control mice exhibited significant improvement in performance over successive trials. In contrast, the Fgf14^{N-βGal/N-βGal} mice were able to remain on the accelerating rod for only a few seconds and their performance did not improve with increasing numbers of trials (FIG. 5B).

Because performance on the rotorod task may depend on several skills such as coordination, balance, strength or various somatosensory capabilities, the mice were tested on a battery of four sensorimotor tests (Wozniak et al., 1996). The performance of the Fgf14^{N-βGal/N-βGal} mice and littermate controls on the inclined (60°) screen test is shown in FIG. 5C. These data show that both groups were able to remain on the screen for the full 60 second trial period, although the Fgf14^{N-βGal/N-βGal} mice took significantly longer to climb to the top of the inclined screen. The ability of the Fgf14^{N-βGal/N-βGal} mice to remain on the screen for the entire trial suggests that they were not grossly impaired in muscle strength. However, this is a relatively insensitive measure of strength (Brosnan-Watters et al., 1996).

The finding that Fgf14^{N-βGal/N-βGal} mice took longer than controls to climb to the top of the inclined screen suggests that the Fgf14^{N-βGal/N-βGal} mice may have deficiencies in coordination and/or strength, although it is difficult to know which function is primarily responsible for the performance difference. Pertinent to these findings are the results of the inverted screen test (FIG. 5C), which is considered to be a more conventional test of grip strength. Fgf14^{N-βGal/N-βGal} mice were severely impaired on this test and were only able to remain on the inverted screen for a few seconds while control mice stayed on the screen for the entire length of the trial. These results suggest that the grip strength of the Fgf14^{N-βGal/N-βGal} mice was impaired, although disturbances in coordination could further contribute to their poor performance. The Fgf14^{N-βGal/N-βGal} mice were also impaired on the ledge and platform tests (FIGS. 5D & E) in that they were able to maintain their balance on the platform and ledge for significantly less time than control mice. Similarly, although ataxia could account for these results, it is difficult to know the relative contributions of potential disturbances in balance, coordination, and/or strength to these performance deficits.

Group performance differences were also observed on the walking initiation test, in which the Fgf14^{N-βGal/N-βGal} mice showed a significant delay in the initiation of movement (ambulation) out of the outlined square (FIG. 5F). This is an interesting finding given that Fgf14^{N-βGal/N-βGal} and control mice were not found to differ in terms of locomotor activity during a one hour testing session. Although the walking initiation data suggests that the Fgf14^{N-βGal/N-βGal} mice exhibit delays in initiating movement, previous drug studies in mice suggest that an alteration in emotional reactivity could also account for the delayed ambulation (e.g., Brosnan-Watters et al., 1996). Even though the poor performance of Fgf14^{N-βGal/N-βGal} mice on the ledge and platform tests may be explained in terms of ataxia, we cannot rule out modest deficits in muscle strength as a contributing factor.

At approximately one month of age, Fgf14^{N-βGal/N-βGal} mice develop paroxysmal forelimb clonic spasms with hyperextended hindlimbs. The symptoms occur several times a day and last 7-12 min. Occasionally, episodes have been observed to last 15-20 min. In young adults (1-3 months), the episodes can cause rearing with loss of balance and forelimb tremor. These symptoms are less severe in older mice (>3 month). Because these episodes resembled seizures, Fgf14^{N-βGal/N-βGal} mice were monitored by electroencephalography (EEG). No surface epileptiform EEG patterns that correlated with the episodes of hyperkinetic movement were observed (n=8 episodes in 4 different Fgf14^{N-βGal/N-βGal} mice) suggesting that these episodes were not seizures.

Several types of paroxysmal dyskinesias have been documented in both mouse and human, including paroxysmal kinesigenic choreoathetosis and paroxysmal dystonia (Bhatia, 1999; Vidailhet, 2000). It has been suggested that both hyperkinetic and hypokinetic movement disorders can be caused by lesions in the basal ganglia. Studies of a rodent model of paroxysmal dystonia indicated that a functional disturbance in the basal ganglion system may be the primary etiology of the neuropathology (Gernert et al., 2000; Nobrega et al., 1996; Rehders et al., 2000). Given the prominent expression of Fgf14^{N-βGal/N-βGal} in the basal ganglia system and the lack of epileptiform activity during the dystonic episodes, the spontaneous neurological phenotype in Fgf14^{N-βGal/N-βGal} mice appears more consistent with a form of dystonia than with a seizure.

Integrity of Nigrostriatal Neurons and Expression of FGF14^{N-βGsl} in Striatonigral Pathways

The basal ganglia system contains feedback circuitry that includes dopaminergic nigrostriatal neurons and GABAergic striatonigral and striatopallidal neurons. The biological actions of the neurotransmitter dopamine are mediated by two classes of receptor, the D1 (D1 and D5) class and the D2 (D2, D3 and D4) class. D1 and D2 receptors are the most abundant dopamine receptors in the adult brain (Missale et al., 1998). Stimulatory D1 receptors are expressed on striatonigral neurons and inhibitory D2 receptors are expressed presynaptically on nigrostriatal neurons and postsynaptically on striatopallidal neurons (Gerfen, 1992).

Neuromuscular diseases, such as Parkinson's disease, often involve degeneration of dopaminergic nigrostriatal neurons that project from the substantia nigra pars compacta (SNc) to the striatum (Dawson, 2000; Smith and Kieval, 2000). Immunostaining for tyrosine hydroxylase, the rate limiting enzyme in dopamine synthesis, revealed normal appearing dopaminergic neurons in the SNc and the ventral tegmental area (VTA) (FIGS. 6A and B) at 2.5 months of age, suggesting no degeneration of dopaminergic neurons. Normal TH staining intensity in the striatum suggests that axonal projections of dopaminergic neurons to the striatum are also intact (FIGS. 6D and E). The ventral striatum is involved in regulating locomotor activity (Swanson et al., 1997). Examination of the levels of dopamine and its metabolites 3,4-dihydroxyphenylacetic acid (DOPAC) and homovanillic acid (HVA) in this region demonstrated comparable levels in control and Fgf14^{N-βGal/N-βGal} mice at 8 months of age (FIG. 6F). Normal TH staining and DA levels indicate that in Fgf14^{N-βGal/N-βGal} mice, the dopaminergic neurons are structurally normal, and produce normal amounts of dopamine. This suggests that the deficits in Fgf14^{N-βGal/N-βGal} mice lie downstream of dopaminergic neurons.

Both northern blot (human brain) and in situ hybridization (mouse brain) failed to detect Fgf14 (or βgal) mRNA expression in the substantia nigra (FIG. 2). In contrast, staining for FGF14N-βgal protein showed intense activity in the striatum, globus pallidus and substantia nigra. Comparison of TH and FGF14N-βgal staining patterns in comparable sections of the substantia nigra (FIGS. 6A-C) showed mutually exclusive expression with FGF14N-βgal protein present in the substantia nigra pars reticulata (SNr) and TH in the SNc. Taken together, these data indicate that Fgf14^{NβGal} is not expressed by dopaminergic neurons, but is expressed in the axons of either or both the striatopallidal (D2R) or striatonigral (D1R) pathways.

Fgf14^{N-βGal/N-βGal} Mice Show Abnormal Responses to Dopamine Agonists

Prominent expression of FGF14 in the basal ganglia system and the importance of this part of the brain in regulating movement suggested that Fgf14^{N-βGal/N-βGal} mice could have an aberrant response to dopaminergic agonists. Dopaminergic pathways can be activated by psychostimulants, such as cocaine and amphetamine. Both drugs increase the level of synaptic dopamine and result in hyperactivity in the mouse, rat and human. Cocaine increases the synaptic dopamine level by blocking the function of the dopamine transporter and thus inhibiting the re-uptake of dopamine. Amphetamine causes release of dopamine from presynaptic nerve terminals in addition to inhibiting re-uptake (ones et al., 1998).

To examine the functional integrity of the basal ganglia system, locomotor activity was monitored in Fgf14^{N-βGal/N-βGal} mice that were challenged with cocaine, amphetamine and specific dopamine receptor agonists. At a dosage of 10 mg/kg, cocaine failed to induce locomotor hyperactivity in 9-12 month old male Fgf14^{N-βGal/N-βGal} mice (p<0.01) (FIG. 7A), while causing a significant hyperactivity response in wildtype littermates. The hyperactivity response to amphetamine at a dose of 2 and 4 mg/kg was significantly lower in Fgf14^{N-βGal/N-βGal} mice than in the wildtype littermates (p<0.01) (FIG. 7A).

Dopamine D1 receptor signaling plays a critical role in the hyperactivity response of mice treated with psychostimulants. Dopamine D1 receptor deficient mice completely lack a locomotor response to cocaine (Xu et al., 1994). In addition, dopamine D1 receptor deficient mice are non-responsive to 2 mg/kg amphetamine and show a decreased response to 5 mg/kg of amphetamine (Xu et al., 2000b). Amphetamine appears to be more potent than cocaine in increasing extracellular dopamine concentration. At 2 mg/kg or 4 mg/kg, amphetamine elevates DA efflux by 350% or 750% above baseline, respectively (Butcher et al., 1988). In contrast, cocaine at 20 mg/kg increased synaptic dopamine by only 120% over baseline (Akimoto et al., 1990). The differential response to cocaine and amphetamine likely reflects the amount of DA released in the striatum and indicates that Fgf14^{N-βGal/N-βGal} mice are unresponsive to small increases in extracellular dopamine induced by cocaine, but are able to respond (to a lesser degree) to high concentrations of dopamine induced by amphetamine. The decreased response to psychostimulants in Fgf14^{N-βGal/N-βGal} and D1R−/− mice likely reflects diminished signaling through the stimulatory dopamine D1 receptor-containing neurons (striatonigral pathway). This hypothesis is further supported by the small decrease in the hyperactivity response induced by the D1 receptor specific agonist SKF38393 in Fgf14^{N-βGal/N-βGal} mice compared to that of wildtype mice (p=0.12) (FIG. 7B).

D1 and D2 receptors appear to be largely segregated in different neuronal populations, and only 20-25% of neurons express both receptors (Surmeier et al., 1998). Activation of presynaptic D2 receptors results in decreased DA release and thus decreased locomotor activity whereas activation of postsynaptic D2 receptors (striatopallidal pathway) results in a small increase in locomotor activity. Quinpirole is a D2-class receptor agonist that can have biphasic effects on locomotor activity in rats (Frantz and Van Hartesveldt, 1999). In mice, a dose of 0.5 mg/kg has been shown to decrease locomotor activity (Geter-Douglass et al., 1997). Consistent with these results, quinpirole, at a dose of 0.5 mg/kg, caused a 4.5 fold decrease in locomotor activity in wildtype mice (FIG. 7B). In contrast, Fgf14^{N-βGal/N-βGal} mice showed no significant response to quinpirole (p<0.001) (FIG. 7B). The lack of response to quinpirole suggests that the D2 receptor mediated signaling is impaired in Fgf14^{N-βGal/N-βGal} mice.

Reduced Cocaine Induced c-fos Activation in FGF14 Deficient Mice

In addition to its acute effect on locomotor behavior, cocaine has a potent effect on gene expression in the striatum (Graybiel et al., 1990). Activated dopamine receptors trigger a series of second messenger cascades that result in the expression and activation of immediate early genes, such as c-fos. The induction of c-fos expression occurs 30-40 min after cocaine injection and peaks at 2 hrs (Moratalla et al., 1996a). D1 and D2 receptors have different roles in c-fos induction (Alonso et al., 1999; Le Moine et al., 1997; Robertson and Jian, 1995). Activation of D1 receptors upregulates c-fos expression in the striatum. Activation of D2 receptors down regulates the basal expression of c-fos in the striatum, but paradoxically increases c-fos expression in the globus pallidus. The optimal dose of cocaine for c-fos induction in mice is 40 mg/kg (Moratalla et al., 1996b). At this dose cocaine robustly induced c-fos expression in the striatum of wildtype mice after 2 hrs (FIG. 8A, C and E). In contrast, in Fgf14^{N-βGal/N-βGal} mice, striatal c-fos induction was dramatically reduced (FIG. 8B, D and E) even though tonic seizures were triggered. The difference in c-fos induction between wildtype and Fgf14^{N-βGal/N-βGal} mice is unlikely to be a consequence of the seizure activity because seizures induce c-fos expression in multiple brain regions including hippocampus, olfactory bulb and cerebellum. Also, cocaine induced c-fos expression in the striatum is largely independent of the onset of seizures (Clark et al., 1992). These data suggest that lack of FGF14 specifically impairs immediate early gene induction in response to cocaine in striatal neurons.

Fgf14^{N-βGal/N-βGal} Mice Have a Reduced Threshold to Seizure Induced by Cocaine and PTZ

High doses of cocaine commonly result in epileptic seizure. The sensitivity to cocaine induced seizure is influenced greatly by genetic factors. For example, C57BL/6 mice are more sensitive to cocaine induced seizures than DBA/2 mice (Hain et al., 2000). Activation of dopamine receptors plays a key role in the initiation of cocaine induced seizure. Dopamine D1 receptors have a pro-convulsive effect whereas D2 receptors have an anti-convulsive effect (Ushijima et al., 1995; Waddington, 1993). In wildtype mice (n=10), injection of cocaine (40 mg/kg) did not induce seizures. In contrast, injection of Fgf14^{N-βGal/N-βGal} mice with 40 mg/kg cocaine resulted in brief tonic movements, mostly in the hindlimbs, followed by a period of immobility (n=9 of 9 mice tested). EEG recordings showed epileptiform activity (slowed rhythm and small spikes) associated with the seizure-like symptoms (FIGS. 9A-D) (n=3/genotype). At 80 mg/kg cocaine, both wildtype mice (n=2) and FGF14 deficient mice (n=2) developed tonic-clonic seizures with associated epileptiform spikes in their EEG recordings (FIGS. 9E and F). The EEG changes at 40 mg/kg of cocaine indicate that the cerebral cortex of Fgf14^{N-βGal/N-βGal} mice may be in a hyper-excitable state.

To determine whether the reduced seizure threshold is restricted to increased sensitivity to cocaine versus resulting from a more widespread affect on cortical hypersensitivity, FGF14 deficient mice and their wildtype littermates were treated with pentylenetetrazol (PTZ), a drug commonly used to induce experimental seizure in rodents (Fisher, 1989). At both 75 mg/kg and 100 mg/kg, FGF14 deficient mice showed a significantly shorter latency to PTZ induced seizure (FIG. 9G).

The FGF14 deficient mice show not only a dramatic decreased latency to PTZ induced seizures, but also an increased time to death. Wild type mice die within 15-20 seconds of seizure onset. Surprisingly, FGF-14 deficient mice enter a state of status epilepticus which lasts for hours (FIG. 13). Fgf14(−/−) mice therefore provide a useful model to study cortical hyperexcitability and pharmacological agents that affect cortical hyperexcitability.

Elevated ERK Activation in Response to PTZ Treatment in FGF14 Deficient Mice

Fgf74 is a member of a subfamily of four related genes, Fgfs11-14. The closely related family member FGF12, interacts with the MAP kinase scaffold protein, islet-brain-2 (IB2). Interestingly, in the absence of FGF12, IB2 does not bind to any MAP kinase tested, but in the presence of FGF12, IB2 efficiently binds p38b, suggesting that FGF12 has a role in modulating MAP kinase signaling pathways (Schoorlemmer and Goldfarb, 2001). Binding experiments demonstrate that FGF14 also interacts with IB2, suggesting that FGF14 may also regulate the association of specific MAP kinases with IB2. To determine whether the level of ERK activation is altered in Fgf14^{N-βGal/N-βGal} mice, we examined ERK and phospho-ERK levels in the brains of Fgf14^{N-βGal/N-βGal} mice and their wildtype littermates. Mice were injected intraperitoneally with PBS or with 75 mg/kg of pentylenetetrazol (PTZ). Brain tissue was harvested 90 sec after injection, divided into cerebellar and cortical hemispheres, and homogenized. ERK and phospho-ERK were detected by immunoblotting. Fgf14^{N-βGal/N-βGal} mice and wildtype littermates showed similar levels of ERK and phospho-ERK following injection with PBS in both cerebellar and cerebral hemispheres (FIG. 9H). In contrast, following treatment with PTZ, the relative level of phospho-ERK was elevated 2 fold (n=7, p=0.036) in cerebral hemispheres of Fgf14^{N-βGal/ N-βGal} mice but not in wildtype mice (FIG. 9I). No change in ERK activation was observed in the cerebellum following treatment with PTZ.

Although the inventors are not bound by theory, it is believed that the foregoing neuropharmacological results can be explained as follows:

FGF14 is widely expressed in the developing CNS and in the adult brain. To determine the role of FGF14 in CNS development and in adult brain function, we have disrupted the Fgf14 gene by homologous recombination and inserted the βgalactosidase cDNA in frame into exon 2. Although the Fgf14^{N-βGal} mRNA is expressed in the correct regions of the brain, the FGF14N-βgal protein is trafficked into axonal processes and thus is present in places where the MRNA is not expressed. Fgf14^{N-βGal/N-βGal} mice are viable, fertile and histologically normal but develop severe ataxia and paroxysmal dyskinesia.

Basal Ganglia Thalamocortical Circuitry in Fgf14^{N-βGal/N-βGal} Mice

In current models of basal ganglia circuitry (FIG. 10A), the striatum (caudate-putamen, CPu), receives input from the cerebral cortex and projects through two pathways to the SNr and the internal segment of the globus pallidus (Gpi) (Todd and Perlmutter, 1998). Neurons in the striatonigral pathway express D1 class dopamine receptors (D1R) and provide inhibitory input to the Gpi/SNr. Striatopallidal neurons primarily express D2 class dopamine receptors (D2R) and provide net excitatory input to the Gpi/SNr via inhibitory projections to the external segment of the globus pallidus (Gpe) and subthalamic nucleus (STN). Dopaminergic neurons projecting to both the striatonigral and striatopallidal pathways express presynaptic D2 receptors which inhibit DA release (Missale et al., 1998). The Gpi/SNr integrates input from these pathways and provides an inhibitory signal to the thalamus. The balance of the net input from these two pathways is critical for the planning, initiation and execution of movements (Obeso et al., 2000; Todd and Perlmutter, 1998; Vitek and Giroux, 2000; Wichmann and DeLong, 1996). Human movement disorders such as Parkinson's disease, Huntington's diseases and dystonia are caused by brain lesions that effectively disrupt the balance between these pathways.

Both the striatonigral and striatopallidal pathways appear to be affected in Fgf14^{N-βGal/N-βGal} mice. Two lines of evidence indicate dysfunction of the striatonigral pathway. First, Fgf14^{N-βGal/N-βGal} mice have reduced locomotor hyperactivity in response to elevated synaptic dopamine levels induced by cocaine and amphetamine. In wildtype mice this hyperactivity response is mediated by striatonigral (D1R) activity in synergy with striatopallidal (postsynaptic D2R) activity (FIG. 10B) (Gershanik et al., 1983; Jackson and Westlind-Danielsson, 1994; Missale et al., 1998). Second, Fgf14^{N-βGal/N-βGal} mice show a 30% reduction in the locomotor activity induced by the D1R agonist SKF38393 (FIG. 7B).

Pharmacologic evidence also demonstrates dysfunction in striatopallidal (D2R) mediated signaling. In drug-naive mice, quinpirole elicits a dose dependent decrease in locomotor activity at a dosage between 0.03 and 2.0 mg/kg (Chen et al., 2000; Geter-Douglass et al., 1997; Jones et al., 1999; Wang et al., 2000b). While 0.5 mg/kg quinpirole caused a -4.5 fold decrease in locomotor activity in wildtype mice, 0.5 or 2 mg/kg quinpirole failed to cause locomotor depression in Fgf14^{N-βGal/N-βGal} mice. Locomotor depression in response to quinpirole can be explained by a predominant effect on presynaptic activity in the striatonigral pathway (decreased synaptic dopamine) and postsynaptic activity in the striatopallidal pathway (FIG. 10A). Because net locomotor activity depends on the integration function of the SNr, it appears that the striatopallidal pathway is more severely affected than the striatonigral pathway in Fgf14^{N-βGal/N-βGal} mice (FIG. 10B).

These experiments clearly show functional defects in both the D1 receptor mediated striatonigral pathway and the striatopallidal pathway. The D1R and D2R expressing neurons in both pathways are primarily GABAergic (Graybiel, 1990). Because FGF14 is most likely localized in axons the observed phenotype may actually result from defects in GABA mediated synaptic transmission or some aspect of axonal function such as vesicle trafficking.

Cortical Hyperexcitability in Fgf14^{N-βGal/N-βGal} Mice

We observe paroxysmal hyperkinetic episodes in Fgf14^{N-βGal/N-βGal} mice. Because the striatonigral and striatopallidal pathways are affected to different degrees in Fgf14^{N-βGal/N-βGal} mice, we posit that the net effect is a reduction in excitatory stimulus to the GPi/SNr, subsequent decreased inhibitory input to the thalamus and increased excitatory stimulus to the motor cortex (FIG. 10B). Enhanced cortical excitability in Fgf14^{N-βGal/N-βGal} mice is supported by the increased sensitivity to cocaine-induced seizures. The cortical hyperactivity in Fgf14^{N-βGal/N-βGal} mice resembles hyperkinetic movement disorders in humans such as Huntington's disease, L-dopa induced dyskinesia in Parkinson's disease, and dystonia (Obeso et al., 2000; Vitek and Giroux, 2000; Wichmann and DeLong, 1996). Although elevated excitatory input to the motor cortex is a general theme in hyperkinetic movement disorders, the primary etiologies are heterogeneous. In Huntington's disease (Wichmann and DeLong, 1996), the degeneration of striatal neurons in the striatopallidal pathway results in a dramatic decrease in D2R mediated inhibitory input to the GPe, and subsequent decreased excitatory input to the GPi/SNr. In contrast, in the case of L-dopa induced dyskinesia, over activation of D1R mediated signals and decreased D2R mediated inhibitory input to the GPe is thought to be the primary cause of the pathology (Obeso et al., 2000; Vitek and Giroux, 2000).

Species differences exist between mouse, rats and humans. In the rat the D2 receptor specific agonist quinpirole has a biphasic effect on locomotor activity that is not observed in mice (Geter-Douglass et al., 1997; Halberda et al., 1997). Species differences have also been documented between rat and human. In rat striatum, the level of D1 receptor is higher than the D2 receptor; whereas, in human the two major types of dopamine receptors are expressed at similar levels (Hall et al., 1988). It is therefore possible that the phenotypic consequences of Fgf14 loss of function may differ between these species.

Fgf14^{N-βGal/N-βGal} Mice Model Paroxysmal Dystonia

Dystonia is a heterogeneous group of syndromes that may have etiologies in a variety of neuronal pathways. Although an imbalance in striatonigral and striatopallidal pathways is sufficient to trigger dystonic episodes in humans and rodents, pathology in other parts of the brain can also cause dystonia (Jarman and Warner, 1998; Richter and Loscher, 1998). Because FGF14 is expressed prominently in cerebellar granule cells it is possible that dysfunction of the cerebellum also contributes to both the dystonia and the ataxia phenotypes.

The episodic nature of the involuntary movement phenotype in Fgf14^{N-βGal/N-βGal} mice is similar to the phenotype of the dystonic hamster (dt^{sz}), the only rodent genetic model of primary paroxysmal dystonia (Loscher et al., 1989). The mutation in the dt^{sz} hamster is autosomal recessive and the gene has not been identified. As in the dt^{sz} hamster, bilateral EEG recordings before and during the dystonic episodes in Fgf14^{N-βGal/N-βGal} mice did not reveal any epileptiform abnormalities, despite the generalized motor involvement. Dystonic events in the dt^{sz} hamster can initiate spontaneously or be induced by mild stressful stimuli. The severity of each event is variable but can be categorized into six stages (Rehders et al., 2000). The most frequently observed phenotypes in Fgf14^{N-βGal/N-βGal} mice correspond to the 3^{rd} and 4^{th} stages in the dt^{sz} hamster, in which the animals have hyperextended hindlimbs, twisting movements and a loss of balance. Unlike in the dt^{sz} hamster, the dystonic episodes in Fgf14^{N-βGal/N-βGal} mice are often accompanied by forelimb tremor. Increased net inhibitory striatal output (decreased inhibitory signals to the thalamus) has been suggested as an etiology of the paroxysmal dystonia in the dt^{sz} hamster (Nobrega et al., 1999; Nobrega et al., 1996; Rehders et al., 2000), similar to that postulated for Fgf14^{N-βGal/N-βGal} mice. The latency and severity of the dystonic attacks in the dt^{sz} hamster are significantly reduced by sub-convulsive doses of pentylenetetrazol (Loscher et al., 1989), suggesting that the cortex of the dt^{sz} hamster is at a higher level of excitability.

Lack of c-fos Induction by Cocaine in Fgf14^{N-βGal/N-βGal} Mice

Immediate early gene expression in the striatum can be induced by psychostimulants such as cocaine and amphetamine (Graybiel et al., 1990), and by the neuroleptic drugs, haloperidol and raclopride (Robertson and Jian, 1995). These drugs have opposite effects on locomotor activity, indicating that the c-fos induction is not directly responsive to increases in locomotor activity. A variety of events, such as seizure activity and hypoxia, can trigger immediate early gene induction. Induced expression of the immediate early gene c-fos can be used as a general marker for increased cell metabolism (reviewed in Kovacs, 1998). The expression of c-fos in the striatum is up-regulated by D1 receptor activation and down-regulated by D2 receptor activation (Keefe and Gerfen, 1995). Striatonigral (D1R) activation appears to be essential for c-fos induction in response to cocaine because D1R deficient mice completely lack c-fos induction in response to psychostimulants (Moratalla et al., 1996b). Paradoxically, activation of both D1 and D2 receptors has a synergistic effect on c-fos induction (Keefe and Gerfen, 1995). The loss of c-fos induction in the striatum in Fgf14^{N-βGal/N-βGal} mice likely reflects diminished signaling through both the striatonigral and striatopallidal pathways and possibly loss of synergy between them.

Molecular Function of FGF14

The data presented in this EXAMPLE I and in several recent publications suggest that FGF14 is important for either axonal function, synaptosomal function or neurotransmission. Here we demonstrate neurological defects in mice lacking FGF14. We also show that 70 amino acids, encoded by exon 1b, are sufficient to efficiently target the β-galactosidase protein into axonal projections. These observations demonstrate neuronal function for FGF14 and suggest that FGF14 itself is localized in axonal projections and may therefore function in these locations.

Fgf14 is a member of a subfamily of four related genes, Fgfs11-14. The closely related family member, FGF12, interacts with the MAP kinase scaffold protein, islet-brain-2 (IB2) (Schoorlemmer and Goldfarb, 2001). Interestingly, in the absence of FGF12, IB2 does not bind to any MAP kinase tested, but in the presence of FGF12 efficiently binds p38δ, suggesting that FGF12 has a role in modulating MAP kinase signaling pathways. The region of FGF12 that binds IB2 includes the FGF core domain and the carboxy-terminus of the molecule but does not require the amino terminus (Schoorlemmer and Goldfarb, 2001). Preliminary binding experiments demonstrate that FGF14 also interacts with IB2 but not IB1, suggesting that FGF14 may also regulate the association of specific map kinases with IB2. A second relevant observation identified an interaction between IB2 and the carboxy-terminal domain of kinesin (Verhey et al., 2001). These data suggest that IB2 and therefore FGF12 and FGF14 may be kinesin cargo transported down axons by kinesin motors. However, the amino terminal peptide (exon 1b), axonal trafficking signal of FGF14 is distinct from the IB2 binding domains and therefore may have a unique role in FGF14 axonal trafficking.

### EXAMPLE II

In this EXAMPLE II, the transgenic knockout mouse (FGF14 KO) of EXAMPLE I was evaluated in a learning/memory test. This test evaluates Pavlovian types of conditioning to the context under which the mice are shocked or given an auditory cue (tone or white noise soundburst) that was paired with the shock.

A test was conducted on the conditioned fear task using the FGF14 KO and WT mice after they had been subjected to a series of drug challenges. The mice were run in a newly acquired conditioned fear system which uses a computerized digital image system to automatically quantify freezing behavior. Using this system it was found that there were very large differences between FGF14 KO and WT mice on the contextual cue test (p<0.00005).

The FGF14 KO mice showed significantly less freezing compared to WT mice on 5/8 minutes tested where differences were marginally nonsignificant on 2 out the remaining 3 minutes. The FGF14 KO mice also showed less freezing that WT mice when they were tested on the auditory cue test (p=0.050) during which time they exhibited significantly less freezing during 2/8 minutes tested after the soundburst that was previously paired with shock was turned on continuously for 8 min.

There was no evidence that the groups differed in the amount of freezing exhibited in response to the altered context baseline nor during baseline training when the mice were exposed to the tone/shock (T/S) pairings. However, the differences during training were marginally nonsignificant and there was some evidence that the FGF14 KO mice exhibited less freezing to the second T/S pairing.

The test session was also videotaped so a human observer could go back and quantify the amount of freezing observed. With this method we confirmed the finding concerning the FGF14 KO mice exhibiting significantly less freezing during the auditory cue conditioning (p=0.006) during which time the KO mice exhibited significantly less freezing during 2 out of the 8 minutes that the auditory cue was turned on and differences during one minute were marginally nonsignificant.

However, there was some evidence that the FGF14 KO mice exhibited significantly less freezing during the first minute of the altered context baseline. Importantly, differences on the contextual cue test were marginally nonsignificant (p=0.088) when a human observer was used to judge freezing. The FGF14 KO mice were judged to exhibit significantly less freezing during only 1 out of 8 of the 1-min intervals—differences were marginally nonsignificant for 1 other interval. No differences were observed during training.

In summary, both methods confirmed that the FGF14 KO mice exhibited significantly more freezing during the auditory cue test than WT controls.

### EXAMPLE III

In this EXAMPLE III, synaptic localization of FGF14-1b in Neuroblastoma (Neuro-2a) cells was demonstrated, and lack of FGF14 was shown to cause elevated excitability in the hippocampal dentate gyrus.

Hippocampal Excitability

Mice were decapitated under halothane anesthesia. The brains were quickly removed and placed in cold oxygenated artificial cerebrospinal fluid (ACSF). Horizontal slices (˜500 μm thick =) of combined hippocampus and entorhinal cortex were taken from the ventral brain surface using a vibratome. Slices were incubated at room temperature in a humidified interface chamber for at least one hour. For physiological recording, individual slices were transferred to an interface chamber, where they were continuously superfused with oxygenated artificial cerebrospinal fluid and humified gas (95% O₂, 5% CO₂) at 34-35° C. ACSF contained (in mM): NaCl 124, NaHC0₃26, KCl 2, KH₂PO₄ 1.3, CaCl₂ 2, MgSO₄ 1.0 or 2.0, glucose 10, with a pH of 7.3-7.4

Extracellular field potential recordings were recorded from the dentate gyrus granule cell layer with single-barrel glass microelectrodes filled with 2 M NaCl. A bipolar metal stimulation electrode was placed in the entorhinal cortex to trigger synaptic inputs via the perforant pathway.

Spontaneous activity was recorded continuously with an Axoclamp-2A amplifier in bridge mode and digitized at a sampling frequency of 1 kHz by Axoscope software for subsequent analysis. Post-synaptic responses to increasing stimulation intensity were sampled at 10 kHz and were quantified by the number of population spikes elicited by the lowest intensity stimulus to trigger a population spike. The frequency of observing single or multiple population spikes in control or FGF-14 knock-out mice were compared by Chi-square analysis.

The results are shown in FIGS. 11 and 12.

Various other examples will be apparent to the person skilled in the art after reading the present disclosure without departing from the spirit and scope of the invention. It is intended that all such other examples be included within the scope of this disclosure and the appended claims.

### References

1. Akimoto, K., Hamamura, T., Kazahaya, Y., Akiyama, K., and Otsuki, S. (1990). Enhanced extracellular dopamine level may be the fundamental neuropharmacological basis of cross-behavioral sensitization between methamphetamine and cocaine—an in vivo dialysis study in freely moving rats, Brain Res 507, 344-6.

2. Alonso, R., Gnanadicom, H., Frechin, N., Fournier, M., Le Fur, G., and Soubrie, P. (1999). Blockade of neurotensin receptors suppresses the dopamine D1/D2 synergism on immediate early gene expression in the rat brain, Eur J Neurosci 11, 967-74.

3. Bahn, S., Jones, A., and Wisden, W. (1997). Directing gene expression to cerebellar granule cells using gamma-aminobutyric acid type A receptor alpha6 subunit transgenes, Proc. Natl. Acad. Sci., U S A 94, 9417-21.

4. Bardgett, M. E., Salaris, S. L., Jackson, J. L., Harding, J., and Csernansky, J. G. (1997). The effects of kainic acid lesions on dopaminergic responses to haloperidol and clozapine, Psychopharmacology (Berl) 133, 142-51.

5. Bhatia, K. P. (1999). The paroxysmal dyskinesias, J Neurol 246, 149-55.

6. Boxer, A. L., Moreno, H., Rudy, B., and Ziff, E. B. (1999). FGF-2 potentiates Ca(2+)-dependent inactivation of NMDA receptor currents in hippocampal neurons, J Neurophysiol 82, 3367-77.

7. Brosnan-Watters, G., Wozniak, D. F., Nardi, A., and Olney, J. W. (1996). Acute behavioral effects of MK-801 in the mouse, Pharmacol Biochem Behav 53, 701-11.

8. Butcher, S. P., Fairbrother, I. S., Kelly, J. S., and Arbuthnott, G. W. (1988). Amphetamine-induced dopamine release in the rat striatum: an in vivo microdialysis study, J Neurochem 50, 346-55.

9. Chae, T., Kwon, Y. T., Bronson, R., Dikkes, P., Li, E., and Tsai, L. H. (1997). Mice lacking p35, a neuronal specific activator of Cdk5, display cortical lamination defects, seizures, and adult lethality, Neuron 18, 29-42.

10. Chen, J. F., Beilstein, M., Xu, Y. H., Turner, T. J., Moratalla, R., Standaert, D. G., Aloyo, V. J., Fink, J. S., and Schwarzschild, M. A. (2000). Selective attenuation of psychostimulant-induced behavioral responses in mice lacking A(2A) adenosine receptors, Neuroscience 97, 195-204.

11. Clark, M., Post, R. M., Weiss, S. R., and Nakajima, T. (1992). Expression of c-fos mRNA in acute and kindled cocaine seizures in rats, Brain Res 582, 101-6.

12. Clarke, M. S., and Feeback, D. L. (1996). Mechanical load induces sarcoplasmic wounding and FGF release in differentiated human skeletal muscle cultures, Faseb J. 10, 502-9.

13. Dawson, T. M. (2000). New animal models for Parkinson's disease, Cell 101, 115-8.

14. Dono, R., Texido, G., Dussel, R., Ehmke, H., and Zeller, R. (1998). Impaired Cerebral Cortex Development and Blood Pressure Regulation in Fgf-2-Deficient Mice, Embo J. 17, 4213-4225.

15. Ferguson, I. A., and Johnson, E. J. (1991). Fibroblast growth factor receptor-bearing neurons in the CNS: identification by receptor-mediated retrograde transport., J. Comp. Neurol. 313, 693-706.

16. Frantz, K. J., and Van Hartesveldt, C. (1999). The locomotor effects of quinpirole in rats depend on age and gender, Pharmacol Biochem Behav 64, 821-6.

17. Gerfen, C. R. (1992). The neostriatal mosaic: multiple levels of compartmental organization, Trends Neurosci 15, 133-9.

18. Gernert, M., Hamann, M., Bennay, M., Loscher, W., and Richter, A. (2000). Deficit of striatal parvalbumin-reactive GABAergic interneurons and decreased basal ganglia. output in a genetic rodent model of idiopathic paroxysmal dystonia, J Neurosci 20, 7052-8.

19. Gershanik, O., Heikkila, R. E., and Duvoisin, R. C. (1983). Behavioral correlations of dopamine receptor activation, Neurology 33, 1489-92.

20. Geter-Douglass, B., Katz, J. L., Alling, K., Acri, J. B., and Witkin, J. M. (1997). Characterization of unconditioned behavioral effects of dopamine D3/D2 receptor agonists, J Pharmacol Exp Ther 283, 7-15.

21. Graybiel, A. M., Moratalla, R., and Robertson, H. A. (1990). Amphetamine and cocaine induce drug-specific activation of the c-fos gene in striosome-matrix compartments and limbic subdivisions of the striatum, Proc. Natl. Acad. Sci., U S A 87, 6912-6.

22. Hain, H. S., Crabbe, J. C., Bergeson, S. E., and Belknap, J. K. (2000). Cocaine-induced seizure thresholds: quantitative trait loci detection and mapping in two populations derived from the C57BL/6 and DBA/2 mouse strains, J Pharmacol Exp Ther 293, 180-7.

23. Halberda, J. P., Middaugh, L. D., Gard, B. E., and Jackson, B. P. (1997). DAD1- and DAD2-like agonist effects on motor activity of C57 mice: differences compared to rats, Synapse 26, 81-92.

24. Hall, H., Farde, L., and Sedvall, G. (1988). Human dopamine receptor subtypes—in vitro binding analysis using 3H-SCH 23390 and 3H-raclopride, J Neural Transm 73, 7-21.

25. Hartung, H., Feldman, B., Lovec, H., Coulier, F., Birnbaum, D., and Goldfarb, M. (1997). Murine FGF-12 and FGF-13: expression in embryonic nervous system, connective tissue and heart, Mech. Dev. 64, 31-39.

26. Jackson, D. M., and Westlind-Danielsson, A. (1994). Dopamine receptors: molecular biology, biochemistry and behavioural aspects, Pharmacol Ther 64, 291-370.

27. Jones, A., Korpi, E. R., McKernan, R. M., Pelz, R., Nusser, Z., Makela, R., Mellor, J. R., Pollard, S., Bahn, S., Stephenson, F. A., et al. (1997). Ligand-gated ion channel subunit partnerships: GABAA receptor alpha6 subunit gene inactivation inhibits delta subunit expression, J Neurosci 17, 1350-62.

28. Jones, S. R., Gainetdinov, R. R., Hu, X. T., Cooper, D. C., Wightman, R. M., White, F. J., and Caron, M. G. (1999). Loss of autoreceptor functions in mice lacking the dopamine transporter, Nat Neurosci 2, 649-55.

29. Jones, S. R., Gainetdinov, R. R., Wightman, R. M., and Caron, M. G. (1998). Mechanisms of amphetamine action revealed in mice lacking the dopamine transporter, J Neurosci 18, 1979-86.

30. Keefe, K. A., and Gerfen, C. R. (1995). D1-D2 dopamine receptor synergy in striatum: effects of intrastriatal infusions of dopamine agonists and antagonists on immediate early gene expression, Neuroscience 66, 903-13.

31. Keresztes, M., and Boonstra, J. (1999). Import(ance) of growth factors in(to) the nucleus [Review], J. Cell Biol. 145, 421-424.

32. Kovacs, K. J. (1998). c-Fos as a transcription factor: a stressful (re)view from a functional map, Neurochem Int 33, 287-97.

33. Lalonde, R. (1987). Motor abnormalities in staggerer mutant mice, Exp Brain Res 68, 417-20.

34. Le Moine, C., Svenningsson, P., Fredholm, B. B., and Bloch, B. (1997). Dopamine-adenosine interactions in the striatum and the globus pallidus: inhibition of striatopallidal neurons through either D2 or A2A receptors enhances D1 receptor-mediated effects on c-fos expression, J Neurosci 17, 8038-48.

35. Loscher, W., Fisher, J. E., Jr., Schmidt, D., Fredow, G., Honack, D., and Iturrian, W. B. (1989). The sz mutant hamster: a genetic model of epilepsy or of paroxysmal dystonia?, Mov. Disord. 4,219-32.

36. Martinez, S., Crossley, P. H., Cobos, I., Rubenstein, J. L., and Martin, G. R. (1999). FGF8 induces formation of an ectopic isthmic organizer and isthmocerebellar development via a repressive effect on Otx2 expression, Development 126, 1189-200.

37. Mason, I. J. (1994). The ins and outs of fibroblast growth factors, Cell 78, 547-52.

38. McFarlane, S., Zuber, M. E., and Holt, C. E. (1998). A role for the Fibroblast Growth Factor Receptor in cell fate decisions in the developing vertebrate retina, Development 125, 3967-3975.

39. Missale, C., Nash, S. R., Robinson, S. W., Jaber, M., and Caron, M. G. (1998). Dopamine receptors: from structure to function, Physiol Rev 78, 189-225.

40. Moratalla, R., Elibol, B., Vallejo, M., and Graybiel, A. M. (1996a). Network-level changes in expression of inducible Fos-Jun proteins in the striatum during chronic cocaine treatment and withdrawal, Neuron 17, 147-56.

41. Moratalla, R., Xu, M., Tonegawa, S., and Graybiel, A. M. (1996b). Cellular responses to psychomotor stimulant and neuroleptic drugs are abnormal in mice lacking the D1 dopamine receptor, Proc. Natl. Acad. Sci., USA 93, 14928-33.

42. Munoz-Sanjuan, I., Smallwood, P. M., and Nathans, J. (2000). Isoform diversity among fibroblast growth factor homologous factors is generated by alternative promoter usage and differential splicing, J. Biol. Chem. 275, 2589-2597.

43. Naski, M. C., Colvin, J. S., Coffin, J. D., and Ornitz, D. M. (1998). Repression of hedgehog signaling and BMP4 expression in growth plate cartilage by fibroblast growth factor receptor 3, Development 125, 4977-88.

44. Nobrega, J. N., Gemert, M., Loscher, W., Raymond, R., Belej, T., and Richter, A. (1999). Tyrosine hydroxylase immunoreactivity and [3H]WIN 35,428 binding to the dopamine transporter in a hamster model of idiopathic paroxysmal dystonia, Neuroscience 92, 211-7.

45. Nobrega, J. N., Richter, A., Tozman, N., Jiwa, D., and Loscher, W. (1996). Quantitative autoradiography reveals regionally selective changes in dopamine D1 and D2 receptor binding in the genetically dystonic hamster, Neuroscience 71, 927-37.

46. Obeso, J. A., Rodriguez-Oroz, M. C., Rodriguez, M., DeLong, M. R., and Olanow, C. W. (2000). Pathophysiology of levodopa-induced dyskinesias in Parkinson's disease: problems with the current model, Ann Neurol 47, S22-32; discussion S32-4.

47. Ornitz, D. M., and Itoh, N. (2001). Fibroblast growth factors, Genome Biol 2, REVIEWS3005.

48. Rehders, J. H., Loscher, W., and Richter, A. (2000). Evidence for striatal dopaminergic overactivity in paroxysmal dystonia indicated by microinjections in a genetic rodent model, Neuroscience 97, 267-77.

49. Robertson, G. S., and Jian, M. (1995). D1 and D2 dopamine receptors differentially increase Fos-like immunoreactivity in accumbal projections to the ventral pallidum and midbrain, Neuroscience 64, 1019-34.

50. Schaefer, M. L., Wong, S. T., Wozniak, D. F., Muglia, L. M., Liauw, J. A., Zhuo, M., Nardi, A., Hartman, R. E., Vogt, S. K., Luedke, C. E., et al. (2000). Altered stress-induced anxiety in adenylyl cyclase type VIII-deficient mice, J Neurosci 20, 4809-20.

51. Schoorlemmer, J., and Goldfarb, M. (2001). Fibroblast growth factor homologous factors are intracellular signaling proteins, Curr. Biol. 11, 793-7.

52. Sheehan, D. C., and Hrapchak, B. B. (1980). Theory and practice of Histotechnology, Vol chapter 14, Nerve tissues, 252-266 (Columbus, Ohio, Battelle Press).

53. Smallwood, P. M., Munoz-sanjuan, I., Tong, P., Macke, J. P., Hendry, S. H., Gilbert, D. J., Copeland, N. G., Jenkins, N. A., and Nathans, J. (1996). Fibroblast growth factor (FGF) homologous factors: new members of the FGF family implicated in nervous system development, Proc. Nati. Acad. Sci., U S A 93, 9850-9857.

54. Smith, Y., and Kieval, J. Z. (2000). Anatomy of the dopamine system in the basal ganglia, Trends Neurosci 23, S28-33.

55. Surrneier, D. J., Yan, Z., and Song, W. J. (1998). Coordinated expression of dopamine receptors in neostriatal medium spiny neurons, Adv Pharmacol 42, 1020-3.

56. Swanson, C. J., Heath, S., Stratford, T. R., and Kelley, A. E. (1997). Differential behavioral responses to dopaminergic stimulation of nucleus accumbens subregions in the rat, Pharmacol Biochem Behav 58, 933-45.

57. Todd, R. D., and Perlmutter, J. S. (1998). Mutational and biochemical analysis of dopamine in dystonia: evidence for decreased dopamine D2 receptor inhibition, Mol Neurobiol 16, 13547.

58. Ushijima, I., Carino, M. A., and Horita, A. (1995). Involvement of D1and D2 dopamine systems in the behavioral effects of cocaine in rats, Pharmacol Biochem Behav 52, 737-41.

59. Verhey, K. J., Meyer, D., Deehan, R., Blenis, J., Schnapp, B. J., Rapoport, T. A., and Margolis, B. (2001). Cargo of kinesin identified as JIP scaffolding proteins and associated signaling molecules, J Cell Biol 152, 959-70.

60. Vidailhet, M. (2000). Paroxysmal dyskinesias as a paradigm of paroxysmal movement disorders [In Process Citation], Curr Opin Neurol 13, 457-62.

61. Vitek, J. L., and Giroux, M. (2000). Physiology of hypokinetic and hyperkinetic movement disorders: model for dyskinesia, Ann Neurol 47, S131-40.

62. Waddington, J. L. (1993). D1:D2 dopamine receptor interactions (San Diego, Academic Press).

63. Wang, Q., McEwen, D. G., and Ornitz, D. M. (2000a). Subcellular and developmental expression of alternatively spliced forms of fibroblast growth factor 14, Mech. Dev. 90, 283-287.

64. Wang, Y., Xu, R., Sasaoka, T., Tonegawa, S., Kung, M. P., and Sankoorikal, E. B. (2000b). Dopamine D2 long receptor-deficient mice display alterations in striatum-dependent functions [In Process Citation], J Neurosci 20, 8305-14.

65. Wichmann, T., and DeLong, M. R. (1996). Functional and pathophysiological models of the basal ganglia, Curr. Opin. Neurobiol 6, 751-8.

66. Wozniak, D. F., Olney, J. W., Kettinger, L. d., Price, M., and Miller, J. P. (1990). Behavioral effects of MK-801 in the rat, Psychopharmacology (Berl) 101, 47-56.

67. Xu, J. S., Liu, Z. H., and Ornitz, D. M. (2000a). Temporal and spatial gradients of Fgf8 and Fgf17 regulate proliferation and differentiation of midline cerebellar structures, Development 127, 1833-1843.

68. Xu, M., Guo, Y., Vorhees, C. V., and Zhang; J. (2000b). Behavioral responses to cocaine and amphetamine administration in mice lacking the dopamine D1 receptor, Brain Res 852, 198-207.

69. Xu, M., Hu, X. T., Cooper, D. C., Moratalla, R., Graybiel, A. M., White, F. J., and Tonegawa, S. (1994). Elimination of cocaine-induced hyperactivity and dopamine-mediated neurophysiological effects in dopamine D1 receptor mutant mice [see comments], Cell 79, 945-55.

70. Yamamoto, A., Lucas, J. J., and Hen, R. (2000). Reversal of neuropathology and motor dysfunction in a conditional model of Huntington's disease, Cell 101, 57-66.

71. Yamamoto, S., Mikami, T., Ohbayashi, N., Ohta, M., and Itoh, N. (1998). Structure and expression of a novel isoform of mouse FGF homologous factor (FHF)-4, Bioch. Biophys. Acta 1398,38-41.

72. Fisher, R. S. (1989). Animal Models of the Epilepsies. Brain Res. Brain Res. Rev. 14, 245-278.

73 Jarman, P. R., and Warner, T. T. (1998). The dystonias. J. Med. Genet. 35, 314-318.

74. Richter, A., and Loscher, W. (1998). Pathology of Idiopathic dystonia: findings from genetic animal models. Prog. Neurobiol. 54,633-677.

What is claimed is:

1. A non-human vertebrate animal whose genome comprises an introduced null mutation in the Fgf14 gene, wherein said animal exhibits ataxia and paroxysmal dyskinesia.

2. A non-human vertebrate animal cell line lacking FGF14 protein expression.

3. A transgenic knockout mouse whose genome comprises an introduced null mutation in the Fgf14 gene wherein said mouse exhibits ataxia and paroxysmal hyperkinetic dyskinesia.

4. A transgenic knockout mouse wherein the in vivo activity of FGF14 is disrupted by homologous introduction with an Fgf14N-βGal allele which abolishes FGF14 expression and generates a fusion protein, FGF14N-βgal, between the first exon of FGF14 and 0-galactosidase.

5. A method of making an FGF14 deficient vertebrate animal or cell line comprising disrupting the Fgf14 gene in said vertebrate animal or cell line by homologous recombination.

6. The method of claim 5 in which the Fgf14 gene is disrupted by homologous introduction of an Fgf14N-βGal allele in said gene.

7. The method of claim 6 in which β-galactosidase cDNA is inserted in frame into exon 2.

8. A method of making a homozygous transgenic knockout mouse whose genome comprises a disrupted Fgf14 gene, comprising the steps of:
(A) deleting the second and third exons of the Fgf14 gene and replacing with P-galactosidase by homologous recombination in mouse embryonic stem cells,
(B) introducing said embryonic stem cells into a mouse blastocyst and transplanting said blastocyst into a pseudopregnant mouse,
(C) allowing said blastocyst to develop into a chimeric mouse,
(D) breeding said chimeric mouse to produce offspring, and
(E) screening said offspring to identify a homozygous transgenic knockout mouse whose genome comprises deletion of the second and third exons of the Fgf14 gene, and wherein said mouse exhibits ataxia and paroxysmal hyperkinetic dyskinesia.

9. A method for screening for activity of a test compound toward a CNS disease comprising administering said test compound to a transgenic knockout mouse as defined in any of claims 3 or 4 and assaying said mouse for improved CNS function.

10. The method of claim 9 in which the CNS disease is a movement disorder.

11. The method of claim 9 in which the CNS disease is ataxia.

12. The method of claim 9 in which the CNS disease is paroxysmal dyskinesis.

13. The method of claim 9 in which the test compound is administered orally or parenterally.

14. A method for screening for activity of a test compound toward an Fgf14 deficient cell line comprising assaying said cell line for subcellular localization of FGF14N-βgal in response to treatment with said test compound.


SEQUENCE LISTING (3 sequences)

SEQ ID NO: 1 - DNA, 24 bp, Artificial Sequence
  /note= “synthetic construct”
ctagtttcatgaaatccctatttc

SEQ ID NO: 2 - DNA, 27 bp, Artificial Sequence
  /note= “synthetic construct”
gccttgcctgcaatataacctggtcac

SEQ ID NO: 3 - DNA, 24 bp, Artificial Sequence
  /note= “synthetic construct”
cgttattacgccagctggcgaaag